SEQUENCE LISTING (27 sequences)

SEQ ID NO: 1 - protein, 7 aa, Artificial Sequence
  Chemically synthesized peptide
DRVYIHP

SEQ ID NO: 2 - protein, 12 aa, Artificial Sequence
  Chemically synthesized peptide
DRVYIHPFHLVI

SEQ ID NO: 3 - protein, 7 aa, Artificial Sequence
  Chemically synthesized peptide
XXXXXXX

SEQ ID NO: 4 - protein, 7 aa, Artificial Sequence
  Chemically synthesized peptide
DRXYIHP

SEQ ID NO: 5 - protein, 7 aa, Artificial Sequence
  Chemically synthesized peptide
DRVSIHC

SEQ ID NO: 6 - protein, 7 aa, Artificial Sequence
  Chemically synthesized peptide
DRVXIHC

SEQ ID NO: 7 - protein, 7 aa, Artificial Sequence
  Chemically synthesized peptide
DRVXIHX

SEQ ID NO: 8 - protein, 10 aa, Artificial Sequence
  Chemically synthesized peptide
DRXXIHXFHL

SEQ ID NO: 9 - protein, 8 aa, Artificial Sequence
  Chemically synthesized peptide
DRXXIHXF

SEQ ID NO: 10 - protein, 7 aa, Artificial Sequence
  Chemically synthesized peptide
RXXIHXF

SEQ ID NO: 11 - protein, 6 aa, Artificial Sequence
  Chemically synthesized peptide
XXIHXF

SEQ ID NO: 12 - protein, 7 aa, Artificial Sequence
  Chemically synthesized peptide
DRXXIHX

SEQ ID NO: 13 - protein, 9 aa, Artificial Sequence
  Chemically synthesized peptide
DRXXIHXFH

SEQ ID NO: 14 - protein, 7 aa, Artificial Sequence
  Chemically synthesized peptide
XXXXXXX

SEQ ID NO: 15 - protein, 7 aa, Artificial Sequence
  Chemically synthesized peptide
DRVXIHA

SEQ ID NO: 16 - protein, 7 aa, Artificial Sequence
  Chemically synthesized peptide
DRVAIHA

SEQ ID NO: 17 - protein, 10 aa, Artificial Sequence
  Chemically synthesized peptide
XXXXXXXXXX

SEQ ID NO: 18 - protein, 7 aa, Artificial Sequence
  Chemically synthesized peptide
DRXXIHA

SEQ ID NO: 19 - protein, 7 aa, Artificial Sequence
  Chemically synthesized peptide
DRXAIHA

SEQ ID NO: 20 - protein, 8 aa, Artificial Sequence
  Chemically synthesized peptide
DRXXIHAI

SEQ ID NO: 21 - protein, 8 aa, Artificial Sequence
  Chemically synthesized peptide
DRXAIHAI

SEQ ID NO: 22 - protein, 7 aa, Artificial Sequence
  Chemically synthesized peptide
DRVSIHC

SEQ ID NO: 23 - DNA, 2599 bp, Homo sapiens
tttttagtctagggaaagtcattcagtggatgtgatcttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataacgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaaaggcgacttcaggatccttatgtgcacaaaggtgacaatggacgacttcctgacagctcatcatgagatggggcatattcagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagatagagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaaaatatcattaaatgtcaaaactatgactctgttcagaaaaaaaaa

SEQ ID NO: 24 - protein, 805 aa, Homo sapiens [sequence omitted]

SEQ ID NO: 25 - protein, 788 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 26 - protein, 723 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 27 - protein, 598 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

(19) United States
(12) Patent Application Publication
(10) Pub. No.: US 2013/0210726 A1
(43) Pub. Date: Aug. 15, 2013

(54) COMPOSITIONS AND METHODS FOR TREATMENT OF PERIPHERAL VASCULAR DISEASE

(71) Applicant: Tarix Pharmaceuticals Ltd., (US)

(72) Inventor: Richard Franklin, Cambridge, MA (US)

(73) Assignee: TARIX PHARMACEUTICALS LTD., Cambridge, MA (US)

(21) Appl. No.: 13/762,285

(22) Filed: Feb. 7, 2013

Related U.S. Application Data
(60) Provisional application No. 61/597,223, filed on Feb. 10, 2012.
(60) Provisional application No. 61/720,301, filed on Oct. 30, 2012.

(51) Int. Cl. A61K 38/22; A61K 31/4178

(52) U.S. Cl. CPC A61K 38/22; A61K 31/4178
USPC 514/9.7; 514/397

(57) ABSTRACT

The present invention relates to compositions and methods for the treatment of peripheral vascular disease (PVD). In particular, the invention provides compositions and methods for treatment of critical limb ischemia, and related diseases, disorders or conditions, based on the use of angiotensin-(1-7) peptides or functional equivalents, analogs or derivatives, angiotensin-(1-7) receptor agonists, ACE2 and/or ACE2 activators.

### CROSS REFERENCE TO RELATED APPLICATIONS

This application claims priority from U.S. provisional patent application Ser. No. 61/597,223, filed Feb. 10, 2012, and U.S. provisional patent application Ser. No. 61/720,301, filed Oct. 30, 2012, the disclosures of which are hereby incorporated in their entirety.

### SEQUENCE LISTING

The present specification makes reference to a Sequence Listing submitted electronically as an ASCII .txt file named “Sequence Listing” on Feb. 7, 2013. The .txt file was generated on Jan. 31, 2013 and is 39 KB in size. The entire contents of the Sequence Listing are herein incorporated by reference.

### BACKGROUND

Peripheral vascular disease (PVD) is generally characterized by partial or complete obstruction of vasculature outside the heart or brain, and can result from atherosclerosis, inflammatory processes leading to stenosis, embolism, or thrombus formation, among others. Peripheral artery disease (PAD) is a form of PVD in which there is a partial or total blockage of arterial blood supply to various internal organs and/or limbs. Risk factors for PAD include elevated blood cholesterol, diabetes, smoking, hypertension, inactivity, and obesity. About 5% of people over the age of 50 are believed to suffer from PAD. Symptoms of PAD depend upon the location and extent of the blocked arteries. The most common symptom of PAD is intermittent claudication, manifested by pain (usually in the calf) that occurs while walking and dissipates at rest. Over time, as the severity of PAD increases, symptoms appear after a shorter duration of exercise. When PAD becomes more severe, symptoms may include pain and cramps at night, pain or tingling in the feet or toes, pain that is worse when legs are elevated and dissipates when legs are dangled (e.g., over the side of the bed), and ulcers that do not heal. PAD can ultimately reach a stage of critical limb ischemia (CLI), which is generally characterized by skin sores that do not heal, ulcers, gangrene, and/or infections in the extremities. In many cases, amputation may be necessary.

PVD (e.g., PAD) can be treated by lifestyle alterations, medications, angioplasty and related treatments, or surgery. Although these therapies alleviate symptoms, and may even improve survival, none can reverse the disease process and directly repair the lasting damage. Impaired angiogenesis is one of the features of ischemic diseases. The most established target for therapeutic angiogenesis has been VEGF and its receptors. However, clinical trials to alleviate ischemia were disappointing. Thus, treatment of PVD (e.g., PAD such as CLI) or other diseases, disorders or conditions associated with impaired angiogenesis remain a major unmet medical need.

### SUMMARY OF THE INVENTION

The present invention provides, among other things, an improved and more effective treatment of Peripheral vascular disease (PVD), such as critical limb ischemia (CLI), and other diseases, disorders or conditions associated with impaired angiogenesis based on angiotensin-(1-7) peptides or functional equivalents, analogs or derivatives, angiotensin-(1-7) receptor agonists, ACE2 and/or ACE2 activators. The present invention is, in part, based on the unexpected discovery that administration of a short seven amino acid peptide known as Angiotensin (1-7) can effectively restore blood flow in an animal model of hind limb ischemia, improving limb function and decreasing ischemic amputations. This discovery is particularly surprising because, prior to the present invention, it was reported that Angiotensin (1-7) has significant antiangiogenic activity by reducing vascular endothelial growth factor-A, a primary proangiogenic protein (see, Soto-Pantoja D. R. et al., “Angiotensin-(1-7) inhibits tumor angiogenesis in human lung cancer xenografts with a reduction in vascular endothelial growth factor,” Mol. Cancer. Ther., 2009; 8(6):1676-83). However, as described in detail in the Examples section below, the present inventors have successfully demonstrated that administration of an angiotensin peptide having seven amino acids identical to the naturally-occurring Angiotensin (1-7) in an animal hind limb ischemia model has effectively restored blood flow resulting in improved limb function, reduced tissue necrosis and ischemic amputations. Thus, contrary to the previous report, the present invention provides angiotensin-based therapeutics that can be used for stimulating therapeutic angiogenesis and treatment of critical limb ischemia and other diseases, disorders or conditions associated with impaired angiogenesis.

Thus, in one aspect, the present invention provides a method for treating peripheral vascular disease including a step of administering a pharmaceutical composition containing an angiotensin (1-7) peptide to an individual suffering from a peripheral vascular disease characterized by partial or complete blockage of blood flow to one or more tissues outside the heart and brain, wherein the angiotensin (1-7) peptide is administered in a therapeutically effective amount such that at least one symptom or feature of the peripheral vascular disease is reduced in intensity, severity, or frequency, or has delayed onset. As used herein, the term “an angiotensin (1-7) peptide” refers to both naturally-occurring Angiotensin (1-7) and any functional equivalent, analogue or derivative of naturally-occurring Angiotensin (1-7). As used herein, “peptide” and “polypeptide” are interchangeable terms and refer to two or more amino acids bound together by a peptide bond. As used herein, the terms “peptide” and “polypeptide” include both linear and cyclic peptides.

In various embodiments, the angiotensin (1-7) peptide includes the naturally-occurring Angiotensin (1-7) amino acid sequence of Asp¹-Arg²-Val³-Tyr⁴-Ile⁵-His⁶-Pro⁷ (SEQ ID NO:1). In some embodiments, the angiotensin (1-7) peptide is a functional equivalent of naturally-occurring Angiotensin (1-7). In certain embodiments, the functional equivalent is a linear peptide.

In some embodiments, a linear peptide contains a sequence that includes at least four, five or six amino acids, respectively, from the seven amino acids that appear in the naturally-occurring Angiotensin (1-7), where the at least four, five or six amino acids, respectively, maintain their relative positions as they appear in the naturally-occurring Angiotensin (1-7), and each linear peptide further has pro-angiogenic activity. In various embodiments, the at least four, five or six amino acids, respectively, further maintain their relative spacing as they appear in the naturally-occurring Angiotensin (1-7).

In some embodiments, the linear peptide contains 4-25 amino acids (e.g., 4-20, 4-15, 4-14, 4-13, 4-12, 4-11, 4-10, 4-9, 4-8, 4-7 amino acids).

In certain embodiments, the linear peptide is a fragment of the naturally-occurring Angiotensin (1-7). In various embodiments, the linear peptide contains amino acid substitutions, deletions and/or insertions in the naturally-occurring Aangiotensin (1-7). In certain embodiments, the linear peptide has an amino acid sequence of Asp¹-Arg²-Nle³-Tyr⁴-Ile⁵-His⁶-Pro⁷ (SEQ ID NO:4) or an amino acid sequence of Asp¹-Arg²-Val³-Ser⁴-Ile⁵-His⁶-Cys⁷ (SEQ ID NO:5).

In various embodiments, the functional equivalent is a cyclic peptide. In certain embodiments, the cyclic peptide includes a linkage between amino acids. In some embodiments, the linkage is located at residues corresponding to positions Tyr⁴ and Pro⁷ in naturally-occurring Angiotensin (1-7). In certain embodiments, the linkage is a thioether bridge. In various embodiments, the cyclic peptide contains an amino acid sequence otherwise identical to the naturally-occurring Angiotensin (1-7) amino acid sequence of Asp¹-Arg²-Val³-Tyr⁴-Ile⁵-His⁶-Pro⁷ (SEQ ID NO:1) or the cyclic peptide includes a norleucine (Nle) replacing position Val³ in naturally-occurring Angiotensin (1-7). In some embodiments, the cyclic peptide is a 4,7-cyclized angiotensin (1-7) with the following formula:

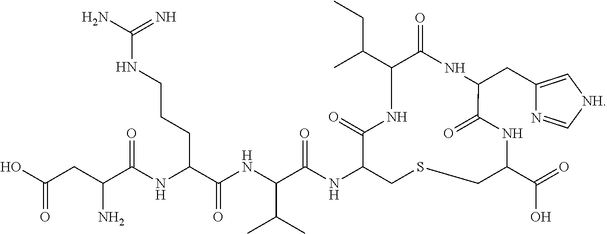 [SMILES: CCC(C)C1NC(=O)C(NC(=O)C(NC(=O)C(CCCNC(=N)N)NC(=O)C(N)CC(=O)O)C(C)C)CSCC(C(=O)O)NC(=O)C(Cc2c[nH]cn2)NC1=O]

In various embodiments, the angiotensin (1-7) peptide contains one or more chemical modifications to increase protease resistance, serum stability and/or bioavailability. In some embodiments, the one or more chemical modifications include pegylation.

In certain embodiments, the one or more tissues outside the heart and brain include one or more limbs of the individual.

In various embodiments, the peripheral vascular disease is a peripheral artery disease. In some embodiments, the peripheral artery disease is critical limb ischemia. In certain embodiments, the peripheral vascular disease is an acute ischemia, a chronic ischemia or is diabetic vascular disease. In some embodiments, the diabetic vascular disease is a nephropathy and/or a neuropathy.

In various embodiments, the angiotensin (1-7) peptide induces and/or increases angiogenesis and/or vascularization in the one or more tissues outside the heart and brain. In certain embodiments, the angiotensin (1-7) peptide decreases and/or delays cell death in the one or more tissues outside the heart and brain. In some embodiments, the cell death is apoptotic or necrotic. In certain embodiments, the angiotensin (1-7) peptide increases and/or enhances cell survival in the one or more tissues outside the heart and brain.

In various embodiments, the therapeutically effective amount of the angiotensin (1-7) peptide is sufficient to decrease partial or total blockage of blood flow to the one or more tissues outside the heart and brain. In some embodiments, the therapeutically effective amount of the angiotensin (1-7) peptide is sufficient to decrease or delay tissue damage in the one or more tissues outside the heart and brain. In certain embodiments, the therapeutically effective amount of the angiotensin is sufficient to improve function of the one or more tissues outside the heart and brain.

In some embodiments, the angiotensin (1-7) peptide is administered parenterally. In certain embodiments, the parenteral administration is selected from intravenous, intradermal, inhalation, transdermal (topical), subcutaneous, and/or transmucosal administration. In various embodiments, the angiotensin (1-7) peptide is administered orally. In some embodiments, the angiotensin (1-7) peptide is administered in conjunction with cyclodextrin. In certain embodiments, wherein the angiotensin (1-7) peptide is administered bimonthly, monthly, triweekly, biweekly, weekly, daily, or at variable intervals.

It is contemplated that various embodiments may use different amounts of angiotensin (1-7) peptide. In some embodiments, the angiotensin (1-7) peptide is administered at an effective dose ranging from about 1-1,000 μg/kg/day (e.g., ranging from about 1-900 μg/kg/day, 1-800 μg/kg/day, 1-700 μg/kg/day, 1-600 μg/kg/day, 1-500 μg/kg/day, 1-400 μg/kg/day, 1-300 μg/kg/day, 1-200 μg/kg/day, 1-100 μg/kg/day, 1-90 μg/kg/day, 1-80 μg/kg/day, 1-70 μg/kg/day, 1-60 μg/kg/day, 1-50 μg/kg/day, 1-40 μg/kg/day, 1-30 μg/kg/day, 1-20 μg/kg/day, 1-10 μg/kg/day). In some embodiments, the angiotensin (1-7) peptide is administered at an effective dose ranging from about 1-500 μg/kg/day. In some embodiments, the angiotensin (1-7) peptide is administered at an effective dose ranging from about 1-100 μg/kg/day. In some embodiments, the angiotensin (1-7) peptide is administered at an effective dose ranging from about 1-60 μg/kg/day. In some embodiments, the angiotensin (1-7) peptide is administered at an effective dose selected from about 1, 2, 4, 6, 8, 10, 15, 20, 25, 30, 35, 40, 45, 50, 75, 100, 150, 200, 250, 300, 350, 400, 450, 500, 550, 600, 650, 700, 750, 800, 850, 900, 950, or 1,000 ug/kg/day.

In certain embodiments, a pro-angiogenic agent is administered in combination with the angiotensin (1-7) peptide. In some embodiments, a vascular or endovascular procedure is performed on the one or more tissues outside the heart and brain.

In another aspect, the present invention provides methods for treating peripheral vascular disease using angiotensin-converting enzyme 2 (ACE2). In some embodiments, the present invention provides a method for treating peripheral vascular disease comprising a step of administering a pharmaceutical composition comprising angiotensin-converting enzyme 2 (ACE2) to an individual suffering from a peripheral vascular disease characterized by partial or complete blockage of blood flow to one or more tissues outside the heart and brain. In some embodiments, the ACE2 is administered in a therapeutically effective amount such that at least one symptom or feature of the peripheral vascular disease is reduced in intensity, severity, or frequency, or has delayed onset.

In still another aspect, the present invention provides a method for treating peripheral vascular disease using an activator of angiotensin-converting enzyme 2 (ACE2). In some embodiments, the present invention provides a method for treating peripheral vascular disease comprising a step of administering a pharmaceutical composition comprising an activator of angiotensin-converting enzyme 2 (ACE2) to an individual suffering from a peripheral vascular disease characterized by partial or complete blockage of blood flow to one or more tissues outside the heart and brain. In some embodiments, a suitable activator of ACE2 is diminazene aceturate (DIZE) and/or 1-[(2-dimethylamino) ethyl amino]-4-(hydroxymethyl)-7-[(4-methylphenyl) sulfonyl oxy]-9H-xanthene-9-one (XNT). In some embodiments, an activator of ACE2 is administered in a therapeutically effective amount such that at least one symptom or feature of the peripheral vascular disease is reduced in intensity, severity, or frequency, or has delayed onset.

In yet another aspect, the present invention provides a method for treating peripheral vascular disease using an angiotensin-(1-7) receptor agonist. In some embodiments, the present invention provides a method for treating peripheral vascular disease comprising a step of administering a pharmaceutical composition comprising an angiotensin-(1-7) receptor agonist to an individual suffering from a peripheral vascular disease characterized by partial or complete blockage of blood flow to one or more tissues outside the heart and brain. In some embodiments, a suitable angiotensin-(1-7) receptor agonist has a formula of

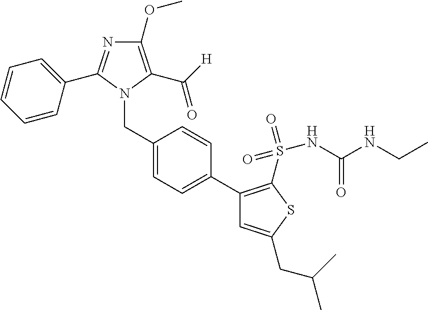 [SMILES: CCNC(=O)NS(=O)(=O)c1sc(CC(C)C)cc1-c1ccc(Cn2c(-c3ccccc3)nc(OC)c2C=O)cc1]

In some embodiments, an angiotensin-(1-7) receptor agonist is administered in a therapeutically effective amount such that at least one symptom or feature of the peripheral vascular disease is reduced in intensity, severity, or frequency, or has delayed onset.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWINGS

The drawings are for illustration purposes only not for limitation.

FIG. 1 depicts exemplary body weight measurements up to 49 days after induction of hindlimb ischemia in mice receiving either TXA127 or a DPBS vehicle.

FIG. 2 depicts exemplary blood flow measurements up to 49 days after induction of hindlimb ischemia in mice receiving either TXA127 or a DPBS vehicle.

FIG. 3 depicts exemplary limb necrosis scores up to 49 days after induction of hindlimb ischemia in mice receiving either TXA127 or a DPBS vehicle.

FIG. 4 depicts exemplary limb amputation dynamics up to 49 days after induction of hindlimb ischemia in mice receiving either TXA127 or a DPBS vehicle.

FIG. 5 depicts exemplary limb functional scores up to 49 days after induction of hindlimb ischemia in mice receiving either TXA127 or a DPBS vehicle.

FIG. 6 depicts exemplary limb functional scores up to 49 days after induction of hindlimb ischemia by using the “last measure carried forward” method of analysis in mice receiving either TXA127 or a DPBS vehicle.

FIG. 7 depicts exemplary body weight measurements up to 49 days after induction of hindlimb ischemia in mice receiving either PanCyte or a DPBS vehicle.

FIG. 8 depicts exemplary blood flow measurements up to 49 days after induction of hindlimb ischemia in mice receiving either PanCyte or a DPBS vehicle.

FIG. 9 depicts exemplary limb functional scores up to 49 days after induction of hindlimb ischemia in mice receiving either PanCyte or a DPBS vehicle.

FIG. 10 depicts exemplary capillary density measurement 49 days after induction of hindlimb ischemia in mice receiving either PanCyte or a DPBS vehicle.

FIG. 11 depicts exemplary body weight measurements up to 49 days after induction of hindlimb ischemia in mice receiving either PanCyte or a DPBS vehicle.

FIG. 12 depicts exemplary blood flow measurements up to 49 days after induction of hindlimb ischemia in mice receiving either PanCyte or a DPBS vehicle.

FIG. 13 depicts exemplary limb functional scores up to 49 days after induction of hindlimb ischemia in mice receiving either PanCyte or a DPBS vehicle.

### DEFINITIONS

In order for the present invention to be more readily understood, certain terms are first defined below. Additional definitions for the following terms and other terms are set forth throughout the specification.

Acute: As used herein, the term “acute” when used in connection with tissue damage and related diseases, disorders, or conditions, has the meaning understood by any one skilled in the medical art. For example, the term typically refers to a disease, disorder, or condition in which there is sudden or severe onset of symptoms. In some embodiments, acute damage is due to an ischemic or traumatic event. Typically, the term “acute” is used in contrast to the term “chronic.”

Agonist: As used herein, the term “agonist” refers to any molecule that has a positive impact in a function of a protein of interest. In some embodiments, an agonist directly or indirectly enhances, strengthens, activates and/or increases an activity of a protein of interest. In particular embodiments, an agonist directly interacts with the protein of interest. Such agonists can be, e.g., proteins, chemical compounds, small molecules, nucleic acids, antibodies, drugs, ligands, or other agents.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion. In certain embodiments, a peptide has no intrinsic biological activity but that inhibits the effects of one or more naturally-occurring angiotensin compounds is considered to be biologically active.

Carrier or diluent: As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution. Exemplary carriers include preparations for tablet or capsule formulation or for inhaled formulations, as discussed in greater detail below.

Chronic: As used herein, the term “chronic,” when used in connection with tissue damage or related diseases, disorders, or conditions has the meaning as understood by any one skilled in the medical art. Typically, the term “chronic” refers to diseases, disorders, or conditions that involve persisting and/or recurring symptoms. Chronic diseases, disorders, or conditions typically develop over a long period of time. The term “chronic” is used in contrast to the term “acute.” In some embodiments, a chronic disease, disorder, or condition results from cell degeneration. In some embodiments, a chronic disease, disorder, or condition results from age-related cell degeneration.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control. In some embodiments, a control is also referred to as a reference.

Critical Limb Ischemia: As used herein, the term “critical limb ischemia” or “CLI” generally refers to a condition characterized by restriction in blood or oxygen supply to the extremities (e.g., hands, feet, legs) of an individual that may result in damage or dysfunction of a tissue in the extremities. Critical limb ischemia may be caused by any of a variety of factors, such as peripheral artery disease (PAD), and may cause severe pain, skin ulcers, or sores, among other symptoms, and in some cases leads to amputation. Critical limb ischemia may be characterized by vasoconstriction, thrombosis, or embolism in one or more extremities. Any tissue in an extremity that normally receives a blood supply can experience critical limb ischemia.

Crude: As used herein, the term “crude,” when used in connection with a biological sample, refers to a sample which is in a substantially unrefined state. For example, a crude sample can be cell lysates or biopsy tissue sample. A crude sample may exist in solution or as a dry preparation.

Diabetic vascular disease: As used herein, the term “diabetic vascular disease” refers to diseases, disorders or conditions associated with the development of blockages in the blood vessels, in particular, arteries because of diabetes. Diabetic vascular disease can be developed throughout the body. In some embodiments, diabetic vascular disease, as used herein, is developed in one or more tissues outside the heart and brain. In some embodiments, diabetic vascular diseases may also include nephropathy (a kidney disease), neuropathy (a condition of the nerves themselves that causes a loss of protective sensation in the toes or feet). Exemplary symptoms of diabetic vascular disease may include, but not be limited to, blurry vision, swelling of face or limbs or unexpected weight gain, foot sores, loss of feeling or a burning feeling in hands or feet, pain in legs when walking, and high blood pressure. A patient suffering from a diabetic vascular disease may eventually develop dead tissue, which is known as gangrene. It can lead to infection and ultimately to amputation.

Dosage form: As used herein, the terms “dosage form” and “unit dosage form” refer to a physically discrete unit of a therapeutic agent for the patient to be treated. Each unit contains a predetermined quantity of active material calculated to produce the desired therapeutic effect. It will be understood, however, that the total dosage of the composition will be decided by the attending physician within the scope of sound medical judgment.

Dysfunction: As used herein, the term “dysfunction” refers to an abnormal function. Dysfunction of a molecule (e.g., a protein) can be caused by an increase or decrease of an activity associated with such molecule. Dysfunction of a molecule can be caused by defects associated with the molecule itself or other molecules that directly or indirectly interact with or regulate the molecule.

Functional equivalent or derivative: As used herein, the term “functional equivalent” or “functional derivative” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved (e.g., it acts as an agonist of Mas receptor). The substituting amino acid desirably has chemico-physical properties which are similar to that of the substituted amino acid. Desirable similar chemico-physical properties include, similarities in charge, bulkiness, hydrophobicity, hydrophilicity, and the like.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same form of disease as the individual being treated, who is about the same age as the individual being treated (to ensure that the stages of the disease in the treated individual and the control individual(s) are comparable).

In vitro: As used herein, the term “in vitro” refers to events that occur in an artificial environment, e.g., in a test tube or reaction vessel, in cell culture, etc., rather than within a multi-cellular organism.

In vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Ischemia: As used herein, the term “ischemia” (also spelled “ischaemia”) typically refers to a restriction in blood or oxygen supply that may result in damage or dysfunction of a tissue. Ischemia may be caused by any of a variety of factors, such as factors in blood vessels, a blood clot, a severe drop in blood pressure, an increase in compartmental pressure, and/or trauma. The term “ischemia” as used herein also refers to local anemia in a given part of a body or tissue that may result, for example, from vasoconstriction, thrombosis, or embolism. Any tissue that normally receives a blood supply can experience ischemia.

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from at least about 10%, about 20%, about 30%, about 40%, about 50%, about 60%, about 70%, about 80%, about 90%, about 95%, about 98%, about 99%, substantially 100%, or 100% of the other components with which they were initially associated. In some embodiments, isolated agents are more than about 80%, about 85%, about 90%, about 91%, about 92%, about 93%, about 94%, about 95%, about 96%, about 97%, about 98%, about 99%, substantially 100%, or 100% pure. As used herein, a substance is “pure” if it is substantially free of other components. As used herein, the term “isolated cell” refers to a cell not contained in a multi-cellular organism.

Peripheral vascular disease: As used herein, the term “peripheral vascular disease” or “PVD” refers to a disease, disorder or condition caused by partial or complete obstruction of blood vessels (e.g., arteries) located outside the heart and brain (e.g., not within the coronary, aortic arch vasculature, or brain). As used herein, the term, “peripheral artery disease” or “PAD” refers to a form of PVD in which there is partial or total blockage of arteries that provide blood supply to one or more tissues located outside the heart and brain (e.g., not within the coronary, aortic arch vasculature, or brain) such as internal organs and/or limbs. As used herein, peripheral vascular disease encompass diabetic vascular disease. See the definition of “diabetic vascular disease.”

Stability: As used herein, the term “stable” refers to the ability of the therapeutic agent to maintain its therapeutic efficacy (e.g., all or the majority of its intended biological activity and/or physiochemical integrity) over extended periods of time. The stability of a therapeutic agent, and the capability of the pharmaceutical composition to maintain stability of such therapeutic agent, may be assessed over extended periods of time (e.g., for at least 1, 3, 6, 12, 18, 24, 30, 36 months or more). In certain embodiments, pharmaceutical compositions described herein have been formulated such that they are capable of stabilizing, or alternatively slowing or preventing the degradation, of one or more therapeutic agents formulated therewith. In the context of a formulation a stable formulation is one in which the therapeutic agent therein essentially retains its physical and/or chemical integrity and biological activity upon storage and during processes (such as freeze/thaw, mechanical mixing and lyophilization).

Subject: As used herein, the term “subject” refers to a human or any non-human animal (e.g., mouse, rat, rabbit, dog, cat, cattle, swine, sheep, horse or primate). A human includes pre and post natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, condition, or event (for example, ischemic stroke) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein associated with the disease, disorder, and/or condition; (4) habits and/or lifestyles associated with development of the disease, disorder, condition, and/or event (5) having undergone, planning to undergo, or requiring a transplant. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent of the invention refers to a peptide inhibitor or derivatives thereof according to the invention.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION OF CERTAIN EMBODIMENTS

The present invention provides, among other things, improved compositions and methods for the treatment of peripheral vascular disease (PVD), such as, critical limb ischemia, and related diseases, disorders or conditions based on the use of angiotensin-(1-7) peptides or functional equivalents, analogs or derivatives, angiotensin-(1-7) receptor agonists, ACE2 and/or ACE2 activators.

Various aspects of the invention are described in detail in the following sections. The use of sections is not meant to limit the invention. Each section can apply to any aspect of the invention. In this application, the use of “or” means “and/or” unless stated otherwise.

### Angiotensin (1-7) Peptides

As used herein, the term “angiotensin (1-7) peptide” refers to both naturally-occurring Angiotensin (1-7) and any functional equivalent, analogue or derivative of naturally-occurring Angiotensin (1-7). As used herein, “peptide” and “polypeptide” are interchangeable terms and refer to two or more amino acids bound together by a peptide bond. As used herein, the terms “peptide” and “polypeptide” include both linear and cyclic peptides. The terms “angiotensin-(1-7)”, “Angiotensin-(1-7)”, and “Ang-(1-7)” are used interchangeably.

Naturally-Occurring Angiotensin (1-7)

Naturally-occurring Angiotensin (1-7) (also referred to as Ang-(1-7)) is a seven amino acid peptide shown below:

(SEQ ID NO: 1)
Asp¹-Arg²-Val³-Tyr⁴-Ile⁵-His⁶-Pro⁷

It is part of the renin-angiotensin system and is converted from a precursor, also known as Angiotensinogen, which is an α-2-globulin that is produced constitutively and released into the circulation mainly by the liver. Angiotensinogen is a member of the serpin family and also known as renin substrate. Human angiotensinogen is 452 amino acids long, but other species have angiotensinogen of varying sizes. Typically, the first 12 amino acids are the most important for angiotensin activity:

(SEQ ID NO: 2)
Asp¹-Arg²-Val³-Tyr⁴-Ile⁵-His⁶-Pro⁷-Phe⁸-His⁹-
Leu¹⁰-Val¹¹-Ile¹²

Different types of angiotensin may be formed by the action of various enzymes. For example, Angiotensin (1-7) is generated by action of Angiotensin-converting enzyme 2 (ACE 2). See the “Angiotensin-converting enzyme 2 (ACE2)” section below.

Ang-(1-7) is an endogenous ligand for Mas receptors. Mas receptors are G-protein coupled receptor containing seven transmembrane spanning regions. As used herein, the term “angiotensin-(1-7) receptor’ encompasses the G Protein-Coupled Mas Receptors.

As used herein, the term “naturally-occurring Angiotensin (1-7)” includes any Angiotensin (1-7) peptide purified from natural sources and any recombinantly produced or chemically synthesized peptides that have an amino acid sequence identical to that of the naturally-occurring Angiotensin (1-7).

Functional Equivalents, Anagloues or Derivatives of Ang-(1-7)

In some embodiments, an angiotensin (1-7) peptide suitable for the present invention is a functional equivalent of naturally-occurring Ang-(1-7). As used herein, a functional equivalent of naturally-occurring Ang-(1-7) refers to any peptide that shares amino acid sequence identity to the naturally-occurring Ang-(1-7) and retain substantially the same or similar activity as the naturally-occurring Ang-(1-7). For example, in some embodiments, a functional equivalent of naturally-occurring Ang-(1-7) described herein has pro-angiogenic activity as determined using methods described herein or known in the art, or an activity such as nitric oxide release, vasodilation, improved endothelial function, antidiuresis, or one of the other properties discussed herein, that positively impacts angiogenesis. In some embodiments, a functional equivalent of naturally-occurring Ang-(1-7) described herein can bind to or activate an angiotensin-(1-7) receptor (e.g., the G protein-coupled Mas receptor) as determined using various assays described herein or known in the art. In some embodiments, a functional equivalent of Ang-(1-7) is also referred to as an angiotensin (1-7) analogue or derivative, or functional derivative.

Typically, a functional equivalent of angiotensin (1-7) shares amino acid sequence similarity to the naturally-occurring Ang-(1-7). In some embodiments, a functional equivalent of Ang-(1-7) according to the invention contains a sequence that includes at least 3 (e.g., at least 4, at least 5, at least 6, at least 7) amino acids from the seven amino acids that appear in the naturally-occurring Ang-(1-7), wherein the at least 3 (e.g., at least 4, at least 5, at least 6, or at least 7) amino acids maintain their relative positions and/or spacing as they appear in the naturally-occurring Ang-(1-7).

In some embodiments, a functional equivalent of Ang-(1-7) also encompass any peptide that contain a sequence at least 50% (e.g., at least 50%, 60, 70%, 80%, or 90%) identical to the amino acid sequence of naturally-occurring Ang-(1-7). Percentage of amino acid sequence identity can be determined by alignment of amino acid sequences. Alignment of amino acid sequences can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzymology 266, 460-480 (1996); http://blast.wustl/edu/blast/README.html). WU-BLAST-2 uses several search parameters, most of which are set to the default values. The adjustable parameters are set with the following values: overlap span=1, overlap fraction=0.125, word threshold (T)=11. HSP score (S) and HSP S2 parameters are dynamic values and are established by the program itself, depending upon the composition of the particular sequence, however, the minimum values may be adjusted and are set as indicated above.

In some embodiments, a functional equivalent, analogue or derivative of Ang-(1-7) is a fragment of the naturally-occurring Ang-(1-7). In some embodiments, a functional equivalent, analogue or derivative of Ang-(1-7) contains amino acid substitutions, deletions and/or insertions in the naturally-occurring Ang-(1-7). Ang-(1-7) functional equivalents, analogues or derivatives can be made by altering the amino acid sequences by substitutions, additions, and/or deletions. For example, one or more amino acid residues within the sequence of the naturally-occurring Ang-(1-7) (SEQ ID NO:1) can be substituted by another amino acid of a similar polarity, which acts as a functional equivalent, resulting in a silent alteration. Substitution for an amino acid within the sequence may be selected from other members of the class to which the amino acid belongs. For example, the positively charged (basic) amino acids include arginine, lysine, and histidine. The nonpolar (hydrophobic) amino acids include leucine, isoleucine, alanine, phenylalanine, valine, proline, tryptophane, and methionine. The uncharged polar amino acids include serine, threonine, cysteine, tyrosine, asparagine, and glutamine. The negatively charged (acid) amino acids include glutamic acid and aspartic acid. The amino acid glycine may be included in either the nonpolar amino acid family or the uncharged (neutral) polar amino acid family. Substitutions made within a family of amino acids are generally understood to be conservative substitutions. For example, the amino acid sequence of a peptide inhibitor can be modified or substituted.

Examples of Ang-(1-7) functional equivalents, analogues and derivatives are described in the section entitled “Exemplary Angiotensin(1-7) Peptides” below.

An angiotensin-(1-7) peptide can be of any length. In some embodiments, an angiotensin-(1-7) peptide according to the present invention can contain, for example, from 4-25 amino acids (e.g., 4-20, 4-15, 4-14, 4-13, 4-12, 4-11, 4-10, 4-9, 4-8, 4-7 amino acids). In some embodiments, an angiotensin-(1-7) peptide according to the present invention can contain from 5-25 amino acid residues, such as 5-20, 5-15 or 5-10 amino acid residues. In some embodiments, an Ang(1-7) peptide according to the present invention contain 4, 5, 6, 7, 8, 9, 10, 11, 12, 13, 14, 15, 16, 17, 18, 19, 20, 21, 22, 23, 24 or 25 residues.

In some embodiments, an angiotensin-(1-7) peptide contains one or more modifications to increase protease resistance, serum stability and/or bioavailability. In some embodiments, suitable modifications are selected from pegylation, acetylation, glycosylation, biotinylation, substitution with D-amino acid and/or un-natural amino acid, and/or cyclization of the peptide.

As used herein, the term “amino acid,” in its broadest sense, refers to any compound and/or substance that can be incorporated into a polypeptide chain. In certain embodiments, an amino acid has the general structure H₂N—C(H)(R)—COOH. In certain embodiments, an amino acid is a naturally-occurring amino acid. In certain embodiments, an amino acid is a synthetic or un-natural amino acid (e.g., α,α-disubstituted amino acids, N-alkyl amino acids); in some embodiments, an amino acid is a D-amino acid; in certain embodiments, an amino acid is an L-amino acid. “Standard amino acid” refers to any of the twenty standard amino acids commonly found in naturally occurring peptides including both L- and D-amino acids which are both incorporated in peptides in nature. “Nonstandard” or “unconventional amino acid” refers to any amino acid, other than the standard amino acids, regardless of whether it is prepared synthetically or obtained from a natural source. As used herein, “synthetic or un-natural amino acid” encompasses chemically modified amino acids, including but not limited to salts, amino acid derivatives (such as amides), and/or substitutions. Amino acids, including carboxy- and/or amino-terminal amino acids in peptides, can be modified by methylation, amidation, acetylation, and/or substitution with other chemical groups that can change the peptide's circulating half-life without adversely affecting its activity. Examples of unconventional or un-natural amino acids include, but are not limited to, citrulline, ornithine, norleucine, norvaline, 4-(E)-butenyl-4(R)-methyl-N-methylthreonine (MeBmt), N-methyl-leucine (MeLeu), aminoisobutyric acid, statine, and N-methyl-alanine (MeAla). Amino acids may participate in a disulfide bond. The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

In certain embodiments, angiotensin-(1-7) peptides contain one or more L-amino acids, D-amino acids, and/or un-natural amino acids.

In addition to peptides containing only naturally occurring amino acids, peptidomimetics or peptide analogs are also encompassed by the present invention. Peptide analogs are commonly used in the pharmaceutical industry as non-peptide drugs with properties analogous to those of the template peptide. The non-peptide compounds are termed “peptide mimetics” or peptidomimetics (Fauchere et al., Infect. Immun. 54:283-287 (1986); Evans et al., J. Med. Chem. 30:1229-1239 (1987)). Peptide mimetics that are structurally related to therapeutically useful peptides may be used to produce an equivalent or enhanced therapeutic or prophylactic effect. Generally, peptidomimetics are structurally similar to the paradigm polypeptide (i.e., a polypeptide that has a biological or pharmacological activity) such as naturally-occurring receptor-binding polypeptides, but have one or more peptide linkages optionally replaced by linkages such as —CH₂NH—, —CH₂S—, —CH₂—CH₂—, —CH═CH— (cis and trans), —CH₂SO—, —CH(OH)CH₂—, —COCH₂— etc., by methods well known in the art (Spatola, Peptide Backbone Modifications, Vega Data, 1(3):267 (1983); Spatola et al. Life Sci. 38:1243-1249 (1986); Hudson et al. Int. J. Pept. Res. 14:177-185 (1979); and Weinstein. B., 1983, Chemistry and Biochemistry, of Amino Acids, Peptides and Proteins, Weinstein eds, Marcel Dekker, New-York,). Such peptide mimetics may have significant advantages over naturally-occurring polypeptides including more economical production, greater chemical stability, enhanced pharmacological properties (e.g., half-life, absorption, potency, efficiency, etc.), reduced antigenicity and others.

Ang-(1-7) peptides also include other types of peptide derivatives containing additional chemical moieties not normally part of the peptide, provided that the derivative retains the desired functional activity of the peptide. Examples of such derivatives include (1) N-acyl derivatives of the amino terminal or of another free amino group, wherein the acyl group may be an alkanoyl group (e.g., acetyl, hexanoyl, octanoyl) an aroyl group (e.g., benzoyl) or a blocking group such as F-moc (fluorenylmethyl-O—CO—); (2) esters of the carboxy terminal or of another free carboxy or hydroxyl group; (3) amide of the carboxy-terminal or of another free carboxyl group produced by reaction with ammonia or with a suitable amine; (4) phosphorylated derivatives; (5) derivatives conjugated to an antibody or other biological ligand and other types of derivatives; and (6) derivatives conjugated to a polyethylene glycol (PEG) chain.

Ang-(1-7) peptides may be obtained by any method of peptide synthesis known to those skilled in the art, including synthetic (e.g., exclusive solid phase synthesis, partial solid phase synthesis, fragment condensation, classical solution synthesis, native-chemical ligation) and recombinant techniques. For example, the peptides or peptides derivatives can be obtained by solid phase peptide synthesis, which in brief, consist of coupling the carboxyl group of the C-terminal amino acid to a resin (e.g., benzhydrylamine resin, chloromethylated resin, hydroxymethyl resin) and successively adding N-alpha protected amino acids. The protecting groups may be any such groups known in the art. Before each new amino acid is added to the growing chain, the protecting group of the previous amino acid added to the chain is removed. Such solid phase synthesis has been disclosed, for example, by Merrifield, J. Am. Chem. Soc. 85: 2149 (1964); Vale et al., Science 213:1394-1397 (1981), in U.S. Pat. Nos. 4,305,872 and 4,316,891, Bodonsky et al. Chem. Ind. (London), 38:1597 (1966); and Pietta and Marshall, Chem. Comm. 650 (1970) by techniques reviewed in Lubell et al. “Peptides” Science of Synthesis 21.11, Chemistry of Amides. Thieme, Stuttgart, 713-809 (2005). The coupling of amino acids to appropriate resins is also well known in the art and has been disclosed in U.S. Pat. No. 4,244,946. (Reviewed in Houver-Weyl, Methods of Organic Chemistry. Vol E22a. Synthesis of Peptides and Peptidomimetics, Murray Goodman, Editor-in-Chief, Thieme. Stuttgart. New York 2002).

Unless defined otherwise, the scientific and technological terms and nomenclature used herein have the same meaning as commonly understood by a person of ordinary skill to which this invention pertains. Generally, the procedures of cell cultures, infection, molecular biology methods and the like are common methods used in the art. Such standard techniques can be found in reference manuals such as, for example, Ausubel et al., Current Protocols in Molecular Biology, Wiley Interscience, New York, 2001; and Sambrook et al., Molecular Cloning: A Laboratory Manual, 3^{rd} edition, Cold Spring Harbor Laboratory Press, N.Y., 2001.

During any process of the preparation of an Ang(1-7) peptide, it may be desirable to protect sensitive reactive groups on any of the molecule concerned. This may be achieved by means of conventional protecting groups such as those described in Protective Groups In Organic Synthesis by T. W. Greene & P. G. M. Wuts, 1991, John Wiley and Sons, New-York; and Peptides: chemistry and Biology by Sewald and Jakubke, 2002, Wiley-VCH, Wheinheim p. 142. For example, alpha amino protecting groups include acyl type protecting groups (e.g., trifluoroacetyl, formyl, acetyl), aliphatic urethane protecting groups (e.g., t-butyloxycarbonyl (BOC), cyclohexyloxycarbonyl), aromatic urethane type protecting groups (e.g., fluorenyl-9-methoxy-carbonyl (Fmoc), benzyloxycarbonyl (Cbz), Cbz derivatives) and alkyl type protecting groups (e.g., triphenyl methyl, benzyl). The amino acids side chain protecting groups include benzyl (for Thr and Ser), Cbz (Tyr, Thr, Ser, Arg, Lys), methyl ethyl, cyclohexyl (Asp, H is), Boc (Arg, His, Cys) etc. The protecting groups may be removed at a convenient subsequent stage using methods known in the art.

Further, Ang-(1-7) peptides may be synthesized according to the FMOC protocol in an organic phase with protective groups. Desirably, the peptides are purified with a yield of 70% with high-pressure liquid chromatography (HPLC) on a C18 chromatography column and eluted with an acetonitrile gradient of 10-60%. The molecular weight of a peptide can be verified by mass spectrometry (reviewed in Fields, G. B. “Solid-Phase Peptide Synthesis” Methods in Enzymology. Vol. 289, Academic Press, 1997).

Alternatively, Ang-(1-7) peptides may be prepared in recombinant systems using, for example, polynucleotide sequences encoding the polypeptides. It is understood that a polypeptide may contain more than one of the above-described modifications within the same polypeptide.

While peptides may be effective in eliciting a biological activity in vitro, their effectiveness in vivo might be reduced by the presence of proteases. Serum proteases have specific substrate requirements. The substrate must have both L-amino acids and peptide bonds for cleavage. Furthermore, exopeptidases, which represent the most prominent component of the protease activity in serum, usually act on the first peptide bond of the peptide and require a free N-terminus (Powell et al., Pharm. Res. 10:1268-1273 (1993)). In light of this, it is often advantageous to use modified versions of peptides. The modified peptides retain the structural characteristics of the original L-amino acid peptides that confer the desired biological activity of Ang-(1-7) but are advantageously not readily susceptible to cleavage by protease and/or exopeptidases.

Systematic substitution of one or more amino acids of a consensus sequence with D-amino acid of the same type (e.g., D-lysine in place of L-lysine) may be used to generate more stable peptides. Thus, a peptide derivative or peptidomimetic of the present invention may be all L, all D or mixed D, L peptide, in either forward or reverse order. The presence of an N-terminal or C-terminal D-amino acid increases the in vivo stability of a peptide since peptidases cannot utilize a D-amino acid as a substrate (Powell et al., Pharm. Res. 10:1268-1273 (1993)). Reverse-D peptides are peptides containing D-amino acids, arranged in a reverse sequence relative to a peptide containing L-amino acids. Thus, the C-terminal residue of an L-amino acid peptide becomes N-terminal for the D-amino acid peptide, and so forth. Reverse D-peptides retain the same secondary conformation and therefore similar activity, as the L-amino acid peptides, but are more resistant to enzymatic degradation in vitro and in vivo, and thus can have greater therapeutic efficacy than the original peptide (Brady and Dodson, Nature 368:692-693 (1994); Jameson et al., Nature 368:744-746 (1994)). Similarly, a reverse-L peptide may be generated using standard methods where the C-terminus of the parent peptide becomes takes the place of the N-terminus of the reverse-L peptide. It is contemplated that reverse L-peptides of L-amino acid peptides that do not have significant secondary structure (e.g., short peptides) retain the same spacing and conformation of the side chains of the L-amino acid peptide and therefore often have the similar activity as the original L-amino acid peptide. Moreover, a reverse peptide may contain a combination of L- and D-amino acids. The spacing between amino acids and the conformation of the side chains may be retained resulting in similar activity as the original L-amino acid peptide.

Another effective approach to confer resistance to peptidases acting on the N-terminal or C-terminal residues of a peptide is to add chemical groups at the peptide termini, such that the modified peptide is no longer a substrate for the peptidase. One such chemical modification is glycosylation of the peptides at either or both termini. Certain chemical modifications, in particular N-terminal glycosylation, have been shown to increase the stability of peptides in human serum (Powell et al., Pharm. Res. 10:1268-1273 (1993)). Other chemical modifications which enhance serum stability include, but are not limited to, the addition of an N-terminal alkyl group, consisting of a lower alkyl of from one to twenty carbons, such as an acetyl group, and/or the addition of a C-terminal amide or substituted amide group. In particular, the present invention includes modified peptides consisting of peptides bearing an N-terminal acetyl group and/or a C-terminal amide group.

Substitution of non-naturally-occurring amino acids for natural amino acids in a subsequence of the peptides can also confer resistance to proteolysis. Such a substitution can, for instance, confer resistance to proteolysis by exopeptidases acting on the N-terminus without affecting biological activity. Examples of non-naturally-occurring amino acids include α,α-disubstituted amino acids, N-alkyl amino acids, C-α-methyl amino acids, β-amino acids, and β-methyl amino acids. Amino acids analogs useful in the present invention may include, but are not limited to, β-alanine, norvaline, norleucine, 4-aminobutyric acid, orithine, hydroxyproline, sarcosine, citrulline, cysteic acid, cyclohexylalanine, 2-aminoisobutyric acid, 6-aminohexanoic acid, t-butylglycine, phenylglycine, o-phosphoserine, N-acetyl serine, N-formylmethionine, 3-methylhistidine and other unconventional amino acids. Furthermore, the synthesis of peptides with non-naturally-occurring amino acids is routine in the art.

In addition, constrained peptides comprising a consensus sequence or a substantially identical consensus sequence variation may be generated by methods well known in the art (Rizo and Gierasch, Ann. Rev. Biochem. 61:387-418 (1992)). For example, constrained peptides may be generated by adding cysteine residues capable of forming disulfide bridges and, thereby, resulting in a cyclic peptide. Cyclic peptides can be constructed to have no free N- or C-termini. Accordingly, they are not susceptible to proteolysis by exopeptidases, although they may be susceptible to endopeptidases, which do not cleave at peptide termini. The amino acid sequences of the peptides with N-terminal or C-terminal D-amino acids and of the cyclic peptides are usually identical to the sequences of the peptides to which they correspond, except for the presence of N-terminal or C-terminal D-amino acid residue, or their circular structure, respectively.

Cyclic Peptides

In some embodiments, a functional equivalent, analogue or derivative of naturally-occurring Ang-(1-7) is a cyclic peptide. As used herein, a cyclic peptide has an intramolecular covalent bond between two non-adjacent residues. The intramolecular bond may be a backbone to backbone, side-chain to backbone or side-chain to side-chain bond (i.e., terminal functional groups of a linear peptide and/or side-chain functional groups of a terminal or interior residue may be linked to achieve cyclization). Typical intramolecular bonds include disulfide, amide and thioether bonds. A variety of means for cyclizing polypeptides are well known in the art, as are many other modifications that can be made to such peptides. For a general discussion, see International Patent Publication Nos. WO 01/53331 and WO 98/02452, the contents of which are incorporated herein by reference. Such cyclic bonds and other modifications can also be applied to the cyclic peptides and derivative compounds of this invention.

Cyclic peptides as described herein may comprise residues of L-amino acids, D-amino acids, or any combination thereof. Amino acids may be from natural or non-natural sources, provided that at least one amino group and at least one carboxyl group are present in the molecule; α- and β-amino acids are generally preferred. Cyclic peptides may also contain one or more rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylation), with or without any of a wide variety of side-chain modifications and/or substitutions (e.g., methylation, benzylation, t-butylation, tosylation, alkoxycarbonylation, and the like). Suitable derivatives include amino acids having an N-acetyl group (such that the amino group that represents the N-terminus of the linear peptide prior to cyclization is acetylated) and/or a C-terminal amide group (i.e., the carboxy terminus of the linear peptide prior to cyclization is amidated). Residues other than common amino acids that may be present with a cyclic peptide include, but are not limited to, penicillamine, β,β-tetramethylene cysteine, β,β-pentamethylene cysteine, β-mercaptopropionic acid, β,β-pentamethylene-β-mercaptopropionic acid, 2-mercaptobenzene, 2-mercaptoaniline, 2-mercaptoproline, ornithine, diaminobutyric acid, α-aminoadipic acid, m-aminomethylbenzoic acid and α,β-diaminopropionic acid.

Following synthesis of a linear peptide, with or without N-acetylation and/or C-amidation, cyclization may be achieved by any of a variety of techniques well known in the art. Within one embodiment, a bond may be generated between reactive amino acid side chains. For example, a disulfide bridge may be formed from a linear peptide comprising two thiol-containing residues by oxidizing the peptide using any of a variety of methods. Within one such method, air oxidation of thiols can generate disulfide linkages over a period of several days using either basic or neutral aqueous media. The peptide is used in high dilution to minimize aggregation and intermolecular side reactions. Alternatively, strong oxidizing agents such as I₂ and K₃Fe(CN)₆ can be used to form disulfide linkages. Those of ordinary skill in the art will recognize that care must be taken not to oxidize the sensitive side chains of Met, Tyr, Trp or His. Within further embodiments, cyclization may be achieved by amide bond formation. For example, a peptide bond may be formed between terminal functional groups (i.e., the amino and carboxy termini of a linear peptide prior to cyclization). Within another such embodiment, the linear peptide comprises a D-amino acid. Alternatively, cyclization may be accomplished by linking one terminus and a residue side chain or using two side chains, with or without an N-terminal acetyl group and/or a C-terminal amide. Residues capable of forming a lactam bond include lysine, ornithine (Orn), α-amino adipic acid, m-aminomethylbenzoic acid, α,β-diaminopropionic acid, glutamate or aspartate. Methods for forming amide bonds are generally well known in the art. Within one such method, carbodiimide-mediated lactam formation can be accomplished by reaction of the carboxylic acid with DCC, DIC, ED AC or DCCI, resulting in the formation of an O-acylurea that can be reacted immediately with the free amino group to complete the cyclization. Alternatively, cyclization can be performed using the azide method, in which a reactive azide intermediate is generated from an alkyl ester via a hydrazide. Alternatively, cyclization can be accomplished using activated esters. The presence of electron withdrawing substituents on the alkoxy carbon of esters increases their susceptibility to aminolysis. The high reactivity of esters of p-nitrophenol, N-hydroxy compounds and polyhalogenated phenols has made these “active esters” useful in the synthesis of amide bonds. Within a further embodiment, a thioether linkage may be formed between the side chain of a thiol-containing residue and an appropriately derivatized α-amino acid. By way of example, a lysine side chain can be coupled to bromoacetic acid through the carbodiimide coupling method (DCC, EDAC) and then reacted with the side chain of any of the thiol containing residues mentioned above to form a thioether linkage. In order to form dithioethers, any two thiol containing side-chains can be reacted with dibromoethane and diisopropylamine in DMF.

### Exemplary Angiotensin-(1-7) Peptides

Linear Angiotensin(1-7) Peptides

In certain aspects, the invention provides linear angiotensin-(1-7) peptides. As discussed above, the structure of naturally-occurring Ang-(1-7) is as follows:

(SEQ ID NO: 1)
Asp¹-Arg²-Val³-Tyr⁴-Ile⁵-His⁶-Pro⁷

The peptides and peptide analogs of the invention can be generally represented by Formula (I):

(SEQ ID NO: 3)
Xaa¹-Xaa²-Xaa³-Xaa⁴-Xaa⁵-Xaa⁶-Xaa⁷,

or a pharmaceutically acceptable salt thereof.

Xaa¹ is any amino acid or a dicarboxylic acid. In certain embodiments, Xaa¹ is Asp, Glu, Asn, Acpc (1-aminocyclopentane carboxylic acid), Ala, Me₂Gly (N,N-dimethylglycine), Pro, Bet (betaine, 1-carboxy-N,N,N-trimethylmethanaminium hydroxide), Glu, Gly, Asp, Sar (sarcosine) or Suc (succinic acid). In certain such embodiments, Xaa¹ is a negatively-charged amino acid, such as Asp or Glu, typically Asp.

Xaa² is Arg, Lys, Ala, Cit (citrulline), Orn (ornithine), acetylated Ser, Sar, D-Arg and D-Lys. In certain embodiments, Xaa² is a positively-charged amino acid such as Arg or Lys, typically Arg.

Xaa³ is Val, Ala, Leu, Nle (norleucine), Ile, Gly, Lys, Pro, HydroxyPro (hydroxyproline), Aib (2-aminoisobutyric acid), Acpc or Tyr. In certain embodiments, Xaa³ is an aliphatic amino acid such as Val, Leu, Ile or Nle, typically Val or Nle.

Xaa⁴ is Tyr, Tyr(PO₃), Thr, Ser, homoSer (homoserine), azaTyr (aza-α¹-homo-L-tyrosine) or Ala. In certain embodiments, Xaa⁴ is a hydroxyl-substituted amino acid such as Tyr, Ser or Thr, typically Tyr.

Xaa⁵ is Ile, Ala, Leu, norLeu, Val or Gly. In certain embodiments, Xaa⁵ is an aliphatic amino acid such as Val, Leu, Ile or Nle, typically Ile.

Xaa⁶ is His, Arg or 6-NH₂-Phe (6-aminophenylalaine). In certain embodiments, Xaa⁶ is a fully or partially positively-charged amino acid such as Arg or His.

Xaa⁷ is Cys, Pro or Ala.

In certain embodiments, one or more of Xaa¹-Xaa⁷ is identical to the corresponding amino acid in naturally-occurring Ang(1-7). In certain such embodiments, all but one or two of Xaa¹-Xaa⁷ are identical to the corresponding amino acid in naturally-occurring Ang(1-7). In other embodiments, all of Xaa¹-Xaa⁶ are identical to the corresponding amino acid in naturally-occurring Ang(1-7).

In certain embodiments, Xaa³ is Nle. When Xaa³ is Nle, one or more of Xaa'-Xaa² and Xaa⁴⁻⁷ are optionally identical to the corresponding amino acid in naturally-occurring Ang(1-7). In certain such embodiments, all but one or two of Xaa¹-Xaa² and Xaa⁴⁻⁷ are identical to the corresponding amino acid in naturally-occurring Ang(1-7). In other embodiments, all of Xaa¹-Xaa² and Xaa⁴⁻⁷ are identical to the corresponding amino acid in naturally-occurring Ang(1-7), resulting in the amino acid sequence: Asp¹-Arg²-Nle³-Tyr⁴-Ile⁵-His⁶-Pro⁷ (SEQ ID NO:4).

In certain embodiments, the peptide has the amino acid sequence Asp¹-Arg²-Nle³-Tyr⁴-Ile⁵-His⁶-Pro⁷ (SEQ ID NO:4).

In certain embodiments, the peptide has the amino acid sequence Asp¹-Arg²-Val³-Ser⁴-Ile⁵-His⁶-Cys (SEQ ID NO:5) or Asp¹-Arg²-Val³-ser⁴-Ile⁵-His⁶-Cys⁷ (SEQ ID NO:6).

Exemplary Cyclic Angiotensin (1-7) Peptides

In certain aspects, the invention provides a cyclic angiotensin-(1-7) (Ang(1-7)) peptide analog comprising a linkage, such as between the side chains of amino acids corresponding to positions Tyr⁴ and Pro⁷ in Ang. These peptide analogs typically comprise 7 amino acid residues, but can also include a cleavable sequence. As discussed in greater detail below, the invention includes fragments and analogs where one or more amino acids are substituted by another amino acid (including fragments). One example of such a fragment or analog is Asp¹-Arg²-Val³-Ser⁴-Ile⁵-His⁶-Cys⁷ (SEQ ID NO:22), wherein a linkage is formed between Ser⁴ and Cys⁷.

Although the following section describes aspects of the invention in terms of a thioether bond linking residues at the 4- and 7-positions, it should be understood that other linkages (as described above) could replace the thioether bridge and that other residues could be cyclized. A thioether bridge is also referred to as a monosulfide bridge or, in the case of Ala-S-Ala, as a lanthionine bridge. Thioether bridge-containing peptides can be formed by two amino acids having one of the following formulas:

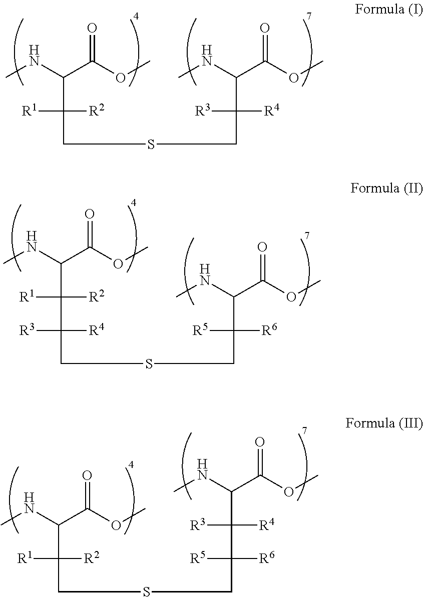

In these formulae, R¹, R², R³, R⁴, R⁵ and R⁶ are independently —H, an alkyl (e.g., C₁-C₆ alkyl, C₁-C₄ alkyl) or an aralkyl group, where the alkyl and aralkyl groups are optionally substituted with one or more halogen, —OH or —NRR′ groups (where R and R′ are independently —H or C₁-C₄ alkyl). In certain embodiments, R¹, R², R³, R⁴, R⁵ and R⁶ are each independently —H or —CH₃, such where all are —H.

In certain embodiments, the invention provides an Ang analog or derivative comprising a thioether bridge according to formula (I). Typically, R¹, R², R³ and R⁴ are independently selected from —H and —CH₃. Peptides comprising a thioether bridge according to formula (I) can be produced, for example, by lantibiotic enzymes or by sulfur extrusion of a disulfide. In one example, the disulfide from which the sulfur is extruded can be formed by D-cysteine in position 4 and L-cysteine in position 7 or by D-cysteine in position 4 and L-penicillamine in position 7 (see, e.g., Galande, Trent and Spatola (2003) Biopolymers 71, 534-551).

In other embodiments, the linkage of the two amino acids can be the bridges depicted in Formula (II) or Formula (III). Peptides comprising a thioether bridge according to Formula (II) can be made, for example, by sulfur extrusion of a disulfide formed by D-homocysteine in position 4 and L-cysteine in position 7. Similarly, peptides comprising a thioether bridge as in Formula (III) can be made, for example, by sulfur extrusion of a disulfide formed by D-cysteine in position 4 and L-homocysteine in position 7.

As discussed above, the Ang analogs and derivatives of the invention vary in length and amino acid composition. The Ang analogs and derivatives of the invention preferably have biological activity or are an inactive precursor molecule that can be proteolytically activated (such as how angiotensin(I), with 10 amino acids, is converted to active fragments by cleavage of 2 amino acids). The size of an Ang analog or derivative can vary but is typically between from about 5 to 10 amino acids, as long as the “core” pentameric segment comprising the 3-7 Nle-thioether-ring structure is encompassed. The amino acid sequence of an analog or derivative of the invention can vary, typically provided that it is biologically active or can become proteolytically activated. Biological activity of an analog or derivative can be determined using methods known in the art, including radioligand binding studies, in vitro cell activation assays and in vivo experiments. See, for example, Godeny and Sayeski, (2006) Am. J. Physiol. Cell. Physiol. 291:C1297-1307; Sarr et al., Cardiovasc. Res. (2006) 71:794-802; and Koziarz et al., (1933) Gen. Pharmacol. 24:705-713.

Ang analogs and derivatives where only the length of the peptide is varied include the following:

a 4,7-cyclized analog designated [Cyc⁴⁻⁷]Ang(1-7), which is derived from natural Ang(1-7) (Asp¹-Arg²-Val³-Cyc⁴-Ile⁵-His⁶-Cyc⁷, SEQ ID NO:7).

a 4,7-cyclized analog designated [Nle³, Cyc⁴⁻⁷]Ang(1-10), which is derived from natural Angiotensin I (Ang(1-10)) (Asp¹-Arg²-Nle³-Cyc⁴-Ile⁵-His⁶-Cyc⁷-Phe⁸-His⁹-Leu¹⁰, SEQ ID NO:8);

a 4,7-cyclized analog designated [Nle³, Cyc⁴⁻⁷]Ang(1-8), which is derived from natural Angiotensin II (Ang(1-8)) (Asp¹-Arg²-Nle³-Cyc⁴-Ile⁵-His⁶-Cyc⁷-Phe⁸, SEQ ID NO:9);

a 4,7-cyclised analog designated [Nle³, Cyc⁴⁻⁷]Ang(2-8), which is derived from natural Angiotensin III (Ang(2-8)) (Arg²-Nle³-Cyc⁴-Ile⁵-His⁶-Cyc⁷-Phe⁸, SEQ ID NO:10);

a 4,7-cyclised analog designated [Nle³, Cyc⁴⁻⁷]Ang(3-8), which is derived from natural Angiotensin IV (Ang(3-8)) (Nle³-Cyc⁴-Ile⁵-His⁶-Cyc⁷-Phe⁸, SEQ ID NO:11);

a 4,7-cyclised analog designated [Nle³, Cyc⁴⁻⁷]Ang(1-7) derived from natural Ang(1-7) (Asp¹-Arg²-Nle³-Cyc⁴-Ile⁵-His⁶-Cyc⁷, SEQ ID NO:12); and

a 4,7-cyclised analog designated [Nle³, Cyc⁴⁻⁷]Ang(1-9) derived from natural Ang(1-9) (Asp¹-Arg²-Nle³-Cyc⁴-Ile⁵-His⁶-Cyc⁷-Phe⁸-His⁹, SEQ ID NO:13).

These analogs can have one of the thioether bridges shown in Formulae (I)-(III) as the Cyc⁴⁻⁷ moiety, for example, where Cyc⁴ and Cyc⁷ are represented by Formula (I), such as where R′—R⁴ are each —H or —CH₃, typically —H.

As compared to the amino acid sequence of the natural angiotensin peptide, the amino acids at positions 4 and 7 of the Cyc⁴⁻⁷ analog are modified to allow introduction of the thioether-ring structures shown above. In addition to the length of the Ang analogs, the amino acids at positions other than 3, 4 and 7 can be the same or different from the naturally-occurring peptide, typically provided that the analog retains a biological function. For analogs of inactive precursors, like [Cyc⁴⁻⁷]Ang(1-10), biological function refers to one or both of an analog's susceptibility to angiotensin-converting enzymes that can cleave it to a biologically active fragment (e.g. Ang(1-8) or Ang(1-7)) or the biological activity of the fragment itself. In certain embodiments, an Ang analog or derivative of the invention has no intrinsic function but inhibits the effects of one or more naturally-occurring angiotensin compounds.

In certain embodiments, an Ang analog of the invention is represented by Formula (IV):

Xaa¹-Xaa²-Xaa³-Cyc⁴-Xaa⁵-Xaa⁶-Cyc⁷(V, SEQ ID NO:14)

Xaa¹ is any amino acid, but typically a negatively-charged amino acid such as Glu or Asp, more typically Asp.

Xaa² is a positively-charged amino acid such as Arg or Lys, typically Arg.

Xaa³ is an aliphatic amino acid, such as Leu, Ile or Val, typically Val.

Cyc⁴ forms a thioether bridge in conjunction with Cyc⁷. Cyc⁴ can be a D-stereoisomer and/or a L-stereoisomer, typically a D-stereoisomer. Examples of Cyc⁴ (taken with Cyc⁷) are shown in Formulas (I), (II) and (III). Typically, the R groups in Formulae (I), (II) and (III) are —H or —CH₃, especially —H.

Xaa⁵ is an aliphatic amino acid, such as Leu, Ile or Val, typically Ile.

Xaa⁶ is His.

Cyc⁷ forms a thioether bridge in conjunction with Cyc⁴, such as in Formula (I), (II) or (III). Cyc⁷ can be a D-stereoisomer and/or a L-stereoisomer, typically a L-stereoisomer. Examples of Cyc⁷ (taken with Cyc⁴) are shown in Formulas (II), (III) and (IV). Typically, the R groups in Formulae (II), (III) and (IV) are —H or —CH₃, especially —H.

In certain embodiments, one or more of Xaa¹-Xaa⁶ (excluding Cyc⁴ and Cyc⁷) is identical to the corresponding amino acid in naturally-occurring Ang-(1-7). In certain such embodiments, all but one or two of Xaa¹-Xaa⁶ are identical to the corresponding amino acid in naturally-occurring Ang-(1-7). In other embodiments, all of Xaa¹-Xaa⁶ are identical to the corresponding amino acid in naturally-occurring Ang-(1-7).

In certain embodiments, Cyc⁴ and Cyc⁷ are independently selected from Abu (2-aminobutyric acid) and Ala (alanine), where Ala is present in at least one position. Thus, cyclic analogs can have a thioether linkage formed by -Ala⁴-S-Ala⁷-(Formula (I), where R¹-R⁴ are each —H); -Ala⁴-S-Abu⁷-(Formula (I): R¹-R³ are —H and R⁴ is —CH₃) or -Abu⁴-S-Ala⁷-(Formula (I): R′, R³ and R⁴ are —H and R² is —CH₃). Specific examples of cyclic analogs comprise a -Abu⁴-S-Ala⁷- or -Ala⁴-S-Ala⁷-linkage.

In certain embodiments, the invention provides an Ang-(1-7) analog with a thioether-bridge between position 4 and position 7 having the amino acid sequence Asp¹-Arg²-Val³-Abu⁴-Ile⁵-His⁶-Ala⁷ (SEQ ID NO:15) or the amino acid sequence Asp¹-Arg²-Val³-Ala⁴-Ile⁵-His⁶-Ala⁷ (SEQ ID NO:16), which are represented by the following structural diagrams:

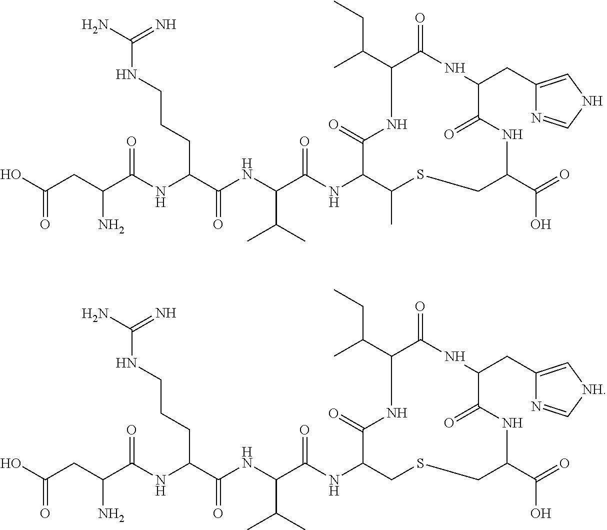 [SMILES: CCC(C)C1NC(=O)C(NC(=O)C(NC(=O)C(CCCNC(=N)N)NC(=O)C(N)CC(=O)O)C(C)C)C(C)SCC(C(=O)O)NC(=O)C(Cc2c[nH]cn2)NC1=O ; CCC(C)C1NC(=O)C(NC(=O)C(NC(=O)C(CCCNC(=N)N)NC(=O)C(N)CC(=O)O)C(C)C)CSCC(C(=O)O)NC(=O)C(Cc2c[nH]cn2)NC1=O]

In certain embodiments, an Ang analog or derivative of the invention is represented by Formula (IV):

Xaa¹-Xaa²-Nle³-Cyc⁴-Xaa⁵-Xaa⁶-Cyc⁷-Xaa⁸-Xaa⁹-Xaa¹⁰(IV, SEQ ID NO:17)

As discussed above, one or more of Xaa', Xaa², Xaa⁸, Xaa⁹ and Xaa¹⁰ are absent in certain embodiments. For example, (1) Xaa¹⁰ is absent, (2) Xaa⁹ and Xaa¹⁰ are absent, (3) Xaa⁸, Xaa⁹ and Xaa¹⁰ are absent, (4) Xaa¹ is absent, (5) Xaa¹ and Xaa¹⁰ are absent, (6) Xaa¹, Xaa⁹ and Xaa¹⁰ are absent, (7) Xaa¹, Xaa⁸, Xaa⁹ and Xaa¹⁰ are absent, (8) Xaa¹ and Xaa² are absent, (9) Xaa¹, Xaa² and Xaa¹⁰ are absent, (10) Xaa¹, Xaa², Xaa⁹ and Xaa¹⁰ are absent, or (11) Xaa¹, Xaa², Xaa⁸, Xaa⁹ and Xaa¹⁰ are absent. For each of these embodiments, the remaining amino acids have the values described below.

Xaa¹, when present, is any amino acid, but typically a negatively charged amino acid such as Glu or Asp, more typically Asp.

Xaa², when present, is a positively charged amino acid such as Arg or Lys, typically Arg.

Nle³ is norleucine.

Cyc⁴ forms a thioether bridge in conjunction with Cyc⁷. Cyc⁴ can be a D-stereoisomer and/or a L-stereoisomer, typically a D-stereoisomer. Examples of Cyc⁴ (taken with Cyc⁷) are shown in Formulas (I), (II) and (III). Typically, the R groups in Formulae (I), (II) and (III) are —H or —CH₃, especially —H.

Xaa⁵ is an aliphatic amino acid, such as Leu, Nle, Ile or Val, typically Ile.

Xaa⁶ is His.

Cyc⁷ forms a thioether bridge in conjunction with Cyc⁴, such as in Formula (I), (II) or (III). Cyc⁷ can be a D-stereoisomer and/or a L-stereoisomer, typically a L-stereoisomer. Examples of Cyc⁷ (taken with Cyc⁴) are shown in Formulas (I), (II) and (III). Typically, the R groups in Formulae (I), (II) and (III) are —H or —CH₃, especially —H.

Xaa⁸, when present, is an amino acid other than Pro, typically Phe or Ile. In certain embodiments, Ile results in an inhibitor of Ang(1-8). In certain embodiments, Phe maintains the biological activity of Ang(1-8) or Ang(1-10).

Xaa⁹, when present, is His.

Xaa¹⁰, when present, is an aliphatic residue, for example, Ile, Val or Leu, typically Leu.

In certain embodiments, one or more of Xaa¹-Xaa¹⁰ (excluding Nle³, Cyc⁴ and Cyc⁷) is identical to the corresponding amino acid in naturally-occurring Ang (including Ang(1-7), Ang(1-8), Ang(1-9), Ang(1-10), Ang(2-7), Ang(2-8), Ang(2-9), Ang(2-10), Ang(3-8), Ang(3-9) and Ang(3-10). In certain such embodiments, all but one or two of Xaa¹-Xaa¹⁰ (for those present) are identical to the corresponding amino acid in naturally-occurring Ang. In other embodiments, all of Xaa¹-Xaa¹⁰ (for those present) are identical to the corresponding amino acid in naturally-occurring Ang.

In certain embodiments, Cyc⁴ and Cyc⁷ are independently selected from Abu (2-aminobutyric acid) and Ala (alanine), where Ala is present at at least one position. Thus, encompassed are cyclic analogs comprising a thioether linkage formed by -Ala⁴-S-Ala⁷-(Formula (I), where R¹-R⁴ are each —H); -Ala⁴-S-Abu⁷-(Formula (I): R′—R³ are —H and R⁴ is —CH₃) or -Abu⁴-S-Ala⁷-(Formula (I): R¹, R³ and R⁴ are —H and R² is —CH₃). Specific cyclic analogs comprise a -Abu⁴-S-Ala⁷- or -Ala⁴-S-Ala⁷-linkage.

In particular, the invention provides an Ang(1-7) analog or derivative with a thioether-bridge between position 4 and position 7 having the amino acid sequence Asp¹-Arg²-Nle³-Abu⁴-Ile⁵-His⁶-Ala⁷ (SEQ ID NO:18) or the amino acid sequence Asp¹-Arg²-Nle³-Ala⁴-Ile⁵-His⁶-Ala⁷ (SEQ ID NO:19).

In another aspect, the invention provides an Ang(1-8) analog or derivative with a thioether-bridge between position 4 and position 7 having Ang(1-8) antagonistic activity, in particular an Ang(1-8) analog or derivative having the amino acid sequence Asp¹-Arg²-Nle³-Abu⁴-Ile⁵-His⁶-Ala⁷-Ile⁸ (SEQ ID NO:20) or the amino acid sequence Asp¹-Arg²-Nle³-Ala⁴-Ile⁵-His⁶-Ala⁷-Ile⁸ (SEQ ID NO:21).

An alkyl group is a straight chained or branched non-aromatic hydrocarbon that is completely saturated. Typically, a straight chained or branched alkyl group has from 1 to about 20 carbon atoms, preferably from 1 to about 10. Examples of straight chained and branched alkyl groups include methyl, ethyl, n-propyl, iso-propyl, n-butyl, sec-butyl, tert-butyl, pentyl, hexyl, pentyl and octyl. A C1-C4 straight chained or branched alkyl group is also referred to as a “lower alkyl” group.

An aralkyl group is an alkyl group substituted by an aryl group. Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrrolyl, pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

### Angiotensin-(1-7) Receptor Agonists

The present invention also contemplates the use of angiotensin-(1-7) receptor agonists in the treatment of peripheral vascular diseases. As used herein, the term “angiotensin-(1-7) receptor agonists” encompasses any molecule that has a positive impact in a function of an angiotensin-(1-7) receptor, in particular, the G-protein coupled Mas receptor. In some embodiments, an angiotensin-(1-7) receptor agonist directly or indirectly enhances, strengthens, activates and/or increases an angiotensin-(1-7) receptor (i.e., the Mas receptor) activity. In some embodiments, an angiotensin-(1-7) receptor agonist directly interacts with an angiotensin-(1-7) receptor (i.e., the Mas receptor). Such agonists can be peptidic or non-peptidic including, e.g., proteins, chemical compounds, small molecules, nucleic acids, antibodies, drugs, ligands, or other agents.

An exemplary class of angiotensin-(1-7) receptor agonists are 1-(p-thienylbenzyl)imidazoles. Examples of these non-peptide angiotensin-(1-7) receptor agonists are represented by Structural Formula (IV):

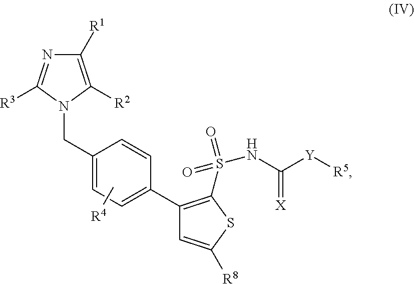

or pharmaceutically acceptable salts thereof, wherein:

R¹ is halogen, hydroxyl, (C₁-C₄)-alkoxy, (C₁-C₈)-alkoxy wherein 1 to 6 carbon atoms are replaced by the heteroatoms O, S, or NH (preferably by O), (C₁-C₄)-alkoxy substituted by a saturated cyclic ether such as tetrahydropyran or tetrahydrofuran, O—(C₁-C₄)-alkenyl, O—(C₁-C₄)-alkylaryl, or aryloxy that is unsubstituted or substituted by a substituent selected from halogen, (C₁-C₃)-alkyl, (C₁-C₃)-alkoxy and trifluoromethyl;

R² is CHO, COOH, or (3) CO—O—(C₁-C₄)-alkyl;

R³ is (C₁-C₄)-alkyl or aryl;

R⁴ is hydrogen, halogen (chloro, bromo, fluoro), or (C₁-C₄)-alkyl;

X is oxygen or sulfur;

Y is oxygen or —NH—;

R⁵ is hydrogen, (C₁-C₆)-alkyl; or (C₁-C₄)-alkylaryl, where R⁵ is hydrogen when Y is —NH—; and

R⁶ is (C₁-C₅)-alkyl.

In certain embodiments, R¹ is not halogen when R² is COOH or CO—O—(C₁-C₄)-alkyl.

In some embodiments, an angiotensin-(1-7) receptor agonist is AVE 0991, 5-formyl-4-methoxy-2-phenyl-1[[4-[2-(ethylaminocarbonylsulfonamido)-5-isobutyl-3-thienyl]-phenyl]-methyl]-imidazole, which is represented by the following structure:

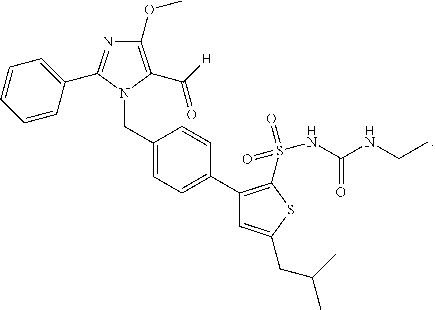 [SMILES: CCNC(=O)NS(=O)(=O)c1sc(CC(C)C)cc1-c1ccc(Cn2c(-c3ccccc3)nc(OC)c2C=O)cc1]

Another exemplary class of angiotensin-(1-7) receptor agonists are p-thienylbenzylamides. Examples of these non-peptide angiotensin-(1-7) receptor agonists are represented by Structural Formula (V):

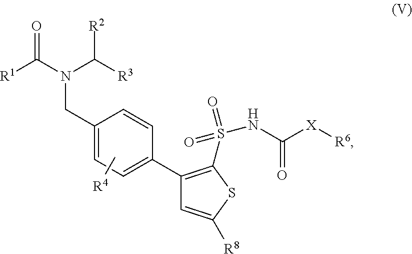

or a pharmaceutically acceptable salt thereof, wherein:

R¹ is (C₁-C₅)-alkyl that is unsubstituted or substituted by a radical chosen from NH₂, halogen, O—(C₁-C₃)-alkyl, CO—O—(C₁-C₃)-alkyl and CO₂H, (C₃-C₈)-cycloalkyl, (C₁-C₃)-alkyl-(C₃-C₈)-cycloalkyl, (C₆-C₁₀)-aryl that is unsubstituted or substituted by a radical chosen from halogen and O—(C₁-C₃)-alkyl, (C₁-C₃)-alkyl-(C₆-C₁₀)-aryl where the aryl radical is unsubstituted or substituted by a radical chosen from halogen and O—(C₁-C₃)-alkyl, (C₁-C₅)-heteroaryl, or (C₁-C₃)-alkyl-(C₁-C₅)-heteroaryl;

R² is hydrogen, (C₁-C₆)-alkyl that is unsubstituted or substituted by a radical chosen from halogen and O—(C₁-C₃)-alkyl, (C₃-C₈)-cycloalkyl, (C₁-C₃)-alkyl-(C₃-C₈)-cycloalkyl, (C₆-C₁₀)-aryl that is unsubstituted or substituted by a radical chosen from among halogen, O—(C₁-C₃)-alkyl and CO—O—(C₁-C₃)-alkyl, or (C₁-C₃)-alkyl-(C₆-C₁₀)-aryl that is unsubstituted or substituted by a radical chosen from halogen and O—(C₁-C₃)-alkyl;

R³ is hydrogen, COOH, or COO—(C₁-C₄)-alkyl;

R⁴ is hydrogen, halogen; or (C₁-C₄)-alkyl;

R⁵ is hydrogen or (C₁-C₆)-alkyl;

R⁶ is hydrogen, (C₁-C₆)-alkyl, (C₁-C₃)-alkyl-(C₃-C₈)-cycloalkyl, or (C₂-C₆)-alkenyl; and

X is oxygen or NH.

Additional examples of angiotensin-(1-7) receptor agonists are described in U.S. Pat. Nos. 6,235,766 and 6,538,144, the contents of which are incorporated by reference herein.

Various angiotensin-(1-7) receptor agonists described above can be present as pharmaceutically acceptable salts. As used herein, “a pharmaceutically acceptable salt” refers to salts that retain the desired activity of the peptide or equivalent compound, but preferably do not detrimentally affect the activity of the peptide or other component of a system, which uses the peptide. Examples of such salts are acid addition salts formed with inorganic acids, for example, hydrochloric acid, hydrobromic acid, sulfuric acid, phosphoric acid, nitric acid, and the like. Salts may also be formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, pamoic acid, alginic acid, polyglutamic acid, and the like. Salts formed from a cationic material may utilize the conjugate base of these inorganic and organic acids. Salts may also be formed with polyvalent metal cations such as zinc, calcium, bismuth, barium, magnesium, aluminum, copper, cobalt, nickel and the like or with an organic cation formed from N,N′-dibenzylethylenediamine or ethylenediamine, or combinations thereof (e.g., a zinc tannate salt). The non-toxic, physiologically acceptable salts are preferred.

The salts can be formed by conventional means such as by reacting the free acid or free base forms of the product with one or more equivalents of the appropriate acid or base in a solvent or medium in which the salt is insoluble, or in a solvent such as water which is then removed in vacuo or by freeze-drying, or by exchanging the cations of an existing salt for another cation on a suitable ion exchange resin.

An alkyl group is a straight chained or branched non-aromatic hydrocarbon that is completely saturated. Typically, a straight chained or branched alkyl group has from 1 to about 20 carbon atoms, preferably from 1 to about 10. Examples of straight chained and branched alkyl groups include methyl, ethyl, n-propyl, iso-propyl, n-butyl, sec-butyl, tert-butyl, pentyl, hexyl, pentyl and octyl. A C₁-C₄ straight chained or branched alkyl group is also referred to as a “lower alkyl” group.

An alkenyl group is a straight chained or branched non-aromatic hydrocarbon that is includes one or more double bonds. Typically, a straight chained or branched alkenyl group has from 2 to about 20 carbon atoms, preferably from 2 to about 10. Examples of straight chained and branched alkenyl groups include ethenyl, n-propenyl, and n-butenyl.

Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrrolyl, pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

An aralkyl group is an alkyl group substituted by an aryl group. Aromatic (aryl) groups include carbocyclic aromatic groups such as phenyl, naphthyl, and anthracyl, and heteroaryl groups such as imidazolyl, thienyl, furyl, pyridyl, pyrimidyl, pyranyl, pyrazolyl, pyrrolyl, pyrazinyl, thiazolyl, oxazolyl, and tetrazolyl. Aromatic groups also include fused polycyclic aromatic ring systems in which a carbocyclic aromatic ring or heteroaryl ring is fused to one or more other heteroaryl rings. Examples include benzothienyl, benzofuryl, indolyl, quinolinyl, benzothiazole, benzoxazole, benzimidazole, quinolinyl, isoquinolinyl and isoindolyl.

### Angiotensin-Converting Enzyme 2 (ACE2)

The present invention also contemplates the use of Angiotensin-converting enzyme 2 (ACE2) in the treatment of peripheral vascular diseases. ACE2 is an enzyme involved in the renin-angiotensin-aldoterone system. ACE2 is generally expressed as a membrane-anchored glycoprotein in various organs, such as heart, kidney, liver and lungs, as well as blood vessels. ACE2 is a carboxypeptidase which cleaves numerous peptide substrates, including apelin, bradykinin, angiotensin I, which is cleaved to angiotensin 1-9, and Ang II, which is cleaved to Ang 1-7. As used herein, the term “ACE2 activity” refers to an ACE2 enzyme or polypeptide that is capable of converting Ang II to Ang 1-7.

Typically, human wild-type ACE2 has 805 amino acid residues, including a signal sequence (amino acids 1-17, underlined in Table 1 below) and a C-terminal hydrophobic end, which is involved in membrane anchoring (bold in Table 1 below). In some embodiments, removal of C-terminal hydrophobic residues leads to an increase in protein solubility. The mRNA and amino acid sequence of human wild-type ACE2 are given in GenBank Accession Nos. AB046569 and BAB40370, respectively, and shown below in Table 1.

TABLE 1
Human ACE2

| | |
|---|---|
| Nucleotide | TTTTTAGTCTAGGGAAAGTCATTCAGTGGATGTGATCTTGGCTCACAGGGGACGATGTCA |
| Sequence (SEQ | AGCTCTTCCTGGCTCCTTCTCAGCCTTGTTGCTGTAACTGCTGCTCAGTCCACCATTGAGG |
| ID NO: 23) | AACAGGCCAAGACATTTTTGGACAAGTTTAACCACGAAGCCGAAGACCTGTTCTATCAAA |
| | GTTCACTTGCTTCTTGGAATTATAACACCAATATTACTGAAGAGAATGTCCAAAACATGA |
| | ATAACGCTGGGGACAAATGGTCTGCCTTTTTAAAGGAACAGTCCACACTTGCCCAAATGT |
| | ATCCACTACAAGAAATTCAGAATCTCACAGTCAAGCTTCAGCTGCAGGCTCTTCAGCAAA |
| | ATGGGTCTTCAGTGCTCTCAGAAGACAAGAGCAAACGGTTGAACACAATTCTAAATACAA |
| | TGAGCACCATCTACAGTACTGGAAAAGTTTGTAACCCAGATAATCCACAAGAATGCTTAT |
| | TACTTGAACCAGGTTTGAATGAAATAATGGCAAACAGTTTAGACTACAATGAGAGGCTCT |
| | GGGCTTGGGAAAGCTGGAGATCTGAGGTCGGCAAGCAGCTGAGGCCATTATATGAAGAG |
| | TATGTGGTCTTGAAAAATGAGATGGCAAGAGCAAATCATTATGAGGACTATGGGGATTAT |
| | TGGAGAGGAGACTATGAAGTAAATGGGGTAGATGGCTATGACTACAGCCGCGGCCAGTT |
| | GATTGAAGATGTGGAACATACCTTTGAAGAGATTAAACCATTATATGAACATCTTCATGC |
| | CTATGTGAGGGCAAAGTTGATGAATGCCTATCCTTCCTATATCAGTCCAATTGGATGCCTC |
| | CCTGCTCATTTGCTTGGTGATATGTGGGGTAGATTTTGGACAAATCTGTACTCTTTGACAG |
| | TTCCCTTTGGACAGAAACCAAACATAGATGTTACTGATGCAATGGTGGACCAGGCCTGGG |
| | ATGCACAGAGAATATTCAAGGAGGCCGAGAAGTTCTTTGTATCTGTTGGTCTTCCTAATAT |
| | GACTCAAGGATTCTGGGAAAATTCCATGCTAACGGACCCAGGAAATGTTCAGAAAGCAGT |
| | CTGCCATCCCACAGCTTGGGACCTGGGGAAAGGCGACTTCAGGATCCTTATGTGCACAAA |
| | GGTGACAATGGACGACTTCCTGACAGCTCATCATGAGATGGGGCATATTCAGTATGATAT |
| | GGCATATGCTGCACAACCTTTTCTGCTAAGAAATGGAGCTAATGAAGGATTCCATGAAGC |
| | TGTTGGGGAAATCATGTCACTTTCTGCAGCCACACCTAAGCATTTAAAATCCATTGGTCTT |
| | CTGTCACCCGATTTTCAAGAAGACAATGAAACAGAAATAAACTTCCTGCTCAAACAAGCA |
| | CTCACGATTGTTGGGACTCTGCCATTTACTTACATGTTAGAGAAGTGGAGGTGGATGGTCT |
| | TTAAAGGGGAAATTCCCAAAGACCAGTGGATGAAAAAGTGGTGGGAGATGAAGCGAGAG |
| | ATAGTTGGGGTGGTGGAACCTGTGCCCCATGATGAAACATACTGTGACCCCGCATCTCTG |
| | TTCCATGTTTCTAATGATTACTCATTCATTCGATATTACACAAGGACCCTTTACCAATTCCA |
| | GTTTCAAGAAGCACTTTGTCAAGCAGCTAAACATGAAGGCCCTCTGCACAAATGTGACAT |
| | CTCAAACTCTACAGAAGCTGGACAGAAACTGTTCAATATGCTGAGGCTTGGAAAATCAGA |
| | ACCCTGGACCCTAGCATTGGAAAATGTTGTAGGAGCAAAGAACATGAATGTAAGGCCACT |
| | GCTCAACTACTTTGAGCCCTTATTTACCTGGCTGAAAGACCAGAACAAGAATTCTTTTGTG |
| | GGATGGAGTACCGACTGGAGTCCATATGCAGACCAAAGCATCAAAGTGAGGATAAGCCT |
| | AAAATCAGCTCTTGGAGATAGAGCATATGAATGGAACGACAATGAAATGTACCTGTTCCG |
| | ATCATCTGTTGCATATGCTATGAGGCAGTACTTTTTAAAAGTAAAAAATCAGATGATTCTT |
| | TTTGGGGAGGAGGATGTGCGAGTGGCTAATTTGAAACCAAGAATCTCCTTTAATTTCTTTG |
| | TCACTGCACCTAAAAATGTGTCTGATATCATTCCTAGAACTGAAGTTGAAAAGGCCATCA |
| | GGATGTCCCGGAGCCGTATCAATGATGCTTTCCGTCTGAATGACAACAGCCTAGAGTTTCT |
| | GGGGATACAGCCAACACTTGGACCTCCTAACCAGCCCCCTGTTTCCATATGGCTGATTGTT |
| | TTTGGAGTTGTGATGGGAGTGATAGTGGTTGGCATTGTCATCCTGATCTTCACTGGGATCA |
| | GAGATCGGAAGAAGAAAAATAAAGCAAGAAGTGGAGAAAATCCTTATGCCTCCATCGAT |
| | ATTAGCAAAGGAGAAAATAATCCAGGATTCCAAAACACTGATGATGTTCAGACCTCCTTT |
| | TAGAAAAATCTATGTTTTTCCTCTTGAGGTGATTTTGTTGTATGTAAATGTTAATTTCATGG |
| | TATAGAAAATATAAGATGATAAAAATATCATTAAATGTCAAAACTATGACTCTGTTCAGA |
| | AAAAAAAA |
| Full-length | MSSSSWLLLSLVAVTAAQSTIEEQAKTFLDKFNHEAEDLFYQSSLASWNYNTNITEENVQNM |
| PrecursorACE2 | NNAGDKWSAFLKEQSTLAQMYPLQEIQNLTVKLQLQALQQNGSSVLSEDKSKRLNTILNTMS |
| Amino Acid | TIYSTGKVCNPDNPQECLLLEPGLNEIMANSLDYNERLWAWESWRSEVGKQLRPLYEEYVVL |
| Sequence (SEQ | KNEMARANHYEDYGDYWRGDYEVNGVDGYDYSRGQLIEDVEHTFEEIKPLYEHLHAYVRA |
| ID NO: 24) | KLMNAYPSYISPIGCLPAHLLGDMWGRFWTNLYSLTVPFGQKPNIDVTDAMVDQAWDAQRIF |
| | KEALKFFVSVGLPNMTQGFWENSMLTDPGNVQKAVCHPTAWDLGKGDFRILMCTKVTMDD |
| | FLTAHHEMGHIQYDMAYAAQPFLLRNGANEGFHEAVGEIMSLSAATPKHLKSIGLLSPDFQED |
| | NETEINFLLKQALTIVGTLPFTYMLEKWRWMVFKGEIPKDQWMKKWWEMKREIVGVVEPVP |
| | HDETYCDPASLFHVSNDYSFIRYYTRTLYQFQFQEALCQAAKHEGPLHKCDISNSTEAGQKLF |
| | NMLRLGKSEPWTLALENVVGAKNMNVRPLLNYFEPLFTWLKDQNKNSFVGWSTDWSPYAD |
| | QSIKVRISLKSALGDRAYEWNDNEMYLFRSSVAYAMRQYFLKVKNQMILFGEEDVRVANLK |
| | PRISFNFFVTAPKNVSDIIPRTEVEKAIRMSRSRINDAFRLNDNSLEFLGIQPTLGPPNQPPVSIWL |
| | IVFGVVMGVIVVGIVILIFTGIRDRKKKNKARSGENPYASIDISKGENNPGFQNTDDVQTSF |
| Full-length | QSTIEEQAKTFLDKFNHEAEDLFYQSSLASWNYNTNITEENVQNMNNAGDKWSAFLKEQSTL |
| Mature ACE2 | AQMYPLQEIQNLTVKLQLQALQQNGSSVLSEDKSKRLNTILNTMSTIYSTGKVCNPDNPQECL |
| Amino Acid | LLEPGLNEIMANSLDYNERLWAWESWRSEVGKQLRPLYEEYVVLKNEMARANHYEDYGDY |
| Sequence (SEQ | WRGDYEVNGVDGYDYSRGQLIEDVEHTFEEIKPLYEHLHAYVRAKLMNAYPSYISPIGCLPA |
| ID NO: 25) | HLLGDMWGRFWTNLYSLTVPFGQKPNIDVTDAMVDQAWDAQRIFKEALKFFVSVGLPNMT |
| | QGFWENSMLTDPGNVQKAVCHPTAWDLGKGDFRILMCTKVTMDDFLTAHHEMGHIQYDM |
| | AYAAQPFLLRNGANEGFHEAVGEIMSLSAATPKHLKSIGLLSPDFQEDNETEINFLLKQALTIV |
| | GTLPFTYMLEKWRWMVFKGEIPKDQWMKKWWEMKREIVGVVEPVPHDETYCDPASLFHVS |
| | NDYSFIRYYTRTLYQFQFQEALCQAAKHEGPLHKCDISNSTEAGQKLFNMLRLGKSEPWTLAL |
| | ENVVGAKNMNVRPLLNYFEPLFTWLKDQNKNSFVGWSTDWSPYADQSIKVRISLKSALGDR |
| | AYEWNDNEMYLFRSSVAYAMRQYFLKVKNQMILFGEEDVRVANLKPRISFNFFVTAPKNVS |
| | DIIPRTEVEKAIRMSRSRINDAFRLNDNSLEFLGIQPTLGPPNQPPVSIWLIVFGVVMGVIVVGI |
| | VILIFTGIRDRKKKNKARSGENPYASIDISKGENNPGFQNTDDVQTSF |
| Mature | QSTIEEQAKTFLDKFNHEAEDLFYQSSLASWNYNTNITEENVQNMNNAGDKWSAFLKEQSTL |
| Truncated | AQMYPLQEIQNLTVKLQLQALQQNGSSVLSEDKSKRLNTILNTMSTIYSTGKVCNPDNPQECL |
| ACE2 Amino | LLEPGLNEIMANSLDYNERLWAWESWRSEVGKQLRPLYEEYVVLKNEMARANHYEDYGDY |
| Acid Sequence | WRGDYEVNGVDGYDYSRGQLIEDVEHTFEEIKPLYEHLHAYVRAKLMNAYPSYISPIGCLPA |
| (SEQ ID | HLLGDMWGRFWTNLYSLTVPFGQKPNIDVTDAMVDQAWDAQRIFKEALKFFVSVGLPNMT |
| NO: 26) | QGFWENSMLTDPGNVQKAVCHPTAWDLGKGDFRILMCTKVTMDDFLTAHHEMGHIQYDM |
| | AYAAQPFLLRNGANEGFHEAVGEIMSLSAATPKHLKSIGLLSPDFQEDNETEINFLLKQALTIV |
| | GTLPFTYMLEKWRWMVFKGEIPKDQWMKKWWEMKREIVGVVEPVPHDETYCDPASLFHVS |
| | NDYSFIRYYTRTLYQFQFQEALCQAAKHEGPLHKCDISNSTEAGQKLFNMLRLGKSEPWTLAL |
| | ENVVGAKNMNVRPLLNYFEPLFTWLKDQNKNSFVGWSTDWSPYADQSIKVRISLKSALGDR |
| | AYEWNDNEMYLFRSSVAYAMRQYFLKVKNQMILFGEEDVRVANLKPRISFNFFVTAPKNVS |
| | DIIPRTEVEKAIRMSRSRINDAFRLNDNSLEFLGIQPTLGPPNQPPVS |
| Mature | QSTIEEQAKTFLDKFNHEAEDLFYQSSLASWNYNTNITEENVQNMNNAGDKWSAFLKEQSTL |
| Truncated | AQMYPLQEIQNLTVKLQLQALQQNGSSVLSEDKSKRLNTILNTMSTIYSTGKVCNPDNPQECL |
| ACE2 Amino | LLEPGLNEIMANSLDYNERLWAWESWRSEVGKQLRPLYEEYVVLKNEMARANHYEDYGDY |
| Acid Sequence | WRGDYEVNGVDGYDYSRGQLIEDVEHTFEEIKPLYEHLHAYVRAKLMNAYPSYISPIGCLPA |
| (SEQ ID | HLLGDMWGRFWTNLYSLTVPFGQKPNIDVTDAMVDQAWDAQRIFKEALKFFVSVGLPNMT |
| NO: 27) | QGFWENSMLTDPGNVQKAVCHPTAWDLGKGDFRILMCTKVTMDDFLTAHHEMGHIQYDM |
| | AYAAQPFLLRNGANEGFHEAVGEIMSLSAATPKHLKSIGLLSPDFQEDNETEINFLLKQALTIV |
| | GTLPFTYMLEKWRWMVFKGEIPKDQWMKKWWEMKREIVGVVEPVPHDETYCDPASLFHVS |
| | NDYSFIRYYTRTLYQFQFQEALCQAAKHEGPLHKCDISNSTEAGQKLFNMLRLGKSEPWTLAL |
| | ENVVGAKNMNVRPLLNYFEPLFTWLKDQNKNSFVGWSTDWSPYAD |

Thus, in some embodiments, an ACE2 enzyme suitable for the present invention is a full length mature human ACE2 protein (SEQ ID NO:25). In some embodiments, an ACE2 enzyme suitable for the present invention is a mature ACE2 enzyme including up to the residue corresponding to amino acid 740 in the full length precursor ACE2 (SEQ ID NO:26). In some embodiments, an ACE2 enzyme suitable for the present invention is a mature ACE2 enzyme including up to the residue corresponding to amino acid 615 in the full length precursor (SEQ ID NO:27). In some embodiments, a suitable ACE2 enzyme may be a homologue or analog of mature human ACE2 enzyme. For example, a homologue or an analogue of mature ACE2 enzyme may be a modified mature human ACE2 enzyme containing one or more amino acid substitutions, deletions, and/or insertions as compared to a wild-type or naturally-occurring ACE2 protein (e.g., SEQ ID NO:25), while retaining substantial ACE2 enzyme activity. Thus, in some embodiments, an ACE2 enzyme suitable for the present invention is substantially homologous to mature human ACE2 protein (SEQ ID NO:25) or protein fragment (SEQ ID NO:26 or SEQ ID NO:27). In some embodiments, an ACE2 enzyme suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO:25. In some embodiments, an ACE2 enzyme suitable for the present invention is substantially identical to mature human ACE2 protein (SEQ ID NO:25) or protein fragment (SEQ ID NO:26 or SEQ ID NO:27). In some embodiments, an ACE2 enzyme suitable for the present invention has an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO:25 or protein fragment (SEQ ID NO:26 or SEQ ID NO:27). In some embodiments, an ACE2 enzyme suitable for the present invention contains a fragment or a portion of mature human ACE2 protein.

Additional examples of ACE2 nucleotide and amino acid sequences are provided in U.S. Publication No. 2011/0020315, U.S. Publication No. 2011/033524, and U.S. Publication No. 2010/0316624, the entire contents of each of which are herein incorporated by reference.

In some embodiments, an ACE2 suitable for the present invention is a fragment of a naturally occurring ACE2 enzyme which retains significant ACE2 activity, i.e., capable of converting Ang II to Ang 1-7. In some embodiments, an ACE2 enzyme suitable for the present invention retains 50%, 60%, 70%, 80%, 85%, 90%, 95%, 98%, 99%, 100% or more activity as compared to wild-type human ACE2 enzyme activity.

In some embodiments, an ACE2 is a soluble form of the ACE2 enzyme. For example, in some embodiments, an ACE2 is a fragment of an ACE2 enzyme that is lacking part or all of the C-terminal hydrophobic region. Solubility of a protein may also be affected by glycosylation. The soluble portion of human wild-type ACE2 has 7 N-glycosylation sites, glycosylation at which sites may increase solubility of the protein. In some embodiments, an ACE2 suitable for the present invention has a glycosylation pattern such that solubility of the protein is increased as compared to a control. In some embodiments, at least 1, 2, 3, 4, 5, 6, or 7 of the ACE2 N-glycosylation sites are glycosylated. In various embodiments, an ACE2 enzyme has a sugar composition of more than 10%, 15%, 20%, or 25% percent by weight of total ACE2. In some embodiments, one or more glycosylation sites are sialysed. For example, in some embodiments, one or more asparagine residues corresponding to position 53, 90, 103, 322, 432, 546 and/or 690 is mono-, di-, tri- or tetra-sialylated. In some embodiments, at least 50%, 60%, 70%, 80%, 90%, 95%, 99% or 100% of the amino acid is either mono-, di-, tri- or tetra-sialylated.

ACE2 is found in all mammals having Ang II as a substrate. It will be appreciated that a suitable ACE2 may be from any organism, including human, mouse, rat, hamster, pig, primate, or cattle, among others.

In some embodiments, ACE2 enzymes are recombinantly produced. Where enzymes are recombinantly produced, any expression system can be used. To give but a few examples, known expression systems include, for example, egg, baculovirus, plant, yeast, or mammalian cells.

In some embodiments, enzymes suitable for the present invention are produced in mammalian cells. Non-limiting examples of mammalian cells that may be used in accordance with the present invention include BALB/c mouse myeloma line (NSO/1, ECACC No: 85110503); human retinoblasts (PER.C6, CruCell, Leiden, The Netherlands); monkey kidney CV1 line transformed by SV40 (COS-7, ATCC CRL 1651); human embryonic kidney line (293 or 293 cells subcloned for growth in suspension culture, Graham et al., J. Gen Virol., 36:59, 1977); human fibrosarcoma cell line (e.g., HT1080); baby hamster kidney cells (BHK, ATCC CCL 10); Chinese hamster ovary cells +/−DHFR (CHO, Urlaub and Chasin, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2).

### ACE2 Activators

The present invention further contemplates the use of ACE2 activators in the treatment of peripheral vascular diseases. As used herein, the term “ACE2 activators” encompasses any molecule that has a positive impact in a function of ACE2. In some embodiments, an ACE2 activator directly or indirectly enhances, strengthens, activates and/or increases an ACE2 activity. In some embodiments, an ACE2 activator directly interacts with ACE2. Such acACE2 activators can be peptidic or non-peptidic. In some embodiments, an ACE2 activator is a small molecule. Various ACE2 activators are known in the art and may be used in accordance with the present invention. For example, diminazene aceturate (DIZE):

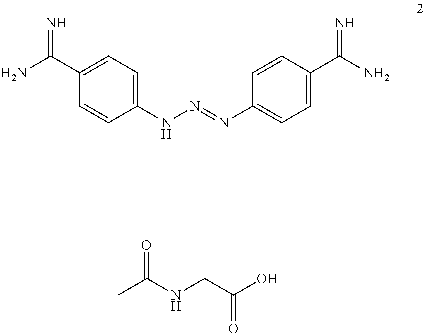

and 1-[(2-dimethylamino) ethyl amino]-4-(hydroxymethyl)-7-[(4-methylphenyl) sulfonyl oxy]-9H-xanthene-9-one (XNT):

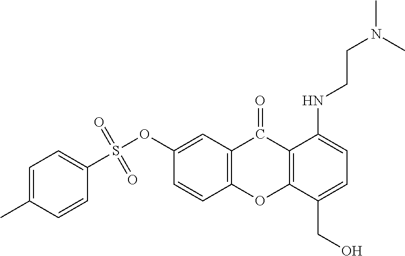

have been shown to function as ACE2 activators; see for example, Gjymishka et al. “Diminazene Aceturate is an ACE2 Activator and a Novel Hypertensive Drug” FASEB J. 24 1032.3 (2010 and Ferreira, et al. “Evidence for Angiotensin-converting Enzyme 2 as a Therapeutic Target for the Prevention of Hypertension” Am. J. Respir. Crit. Care Med. 179:1048 (2009), the entire contents of each of which are herein incorporated by reference. Additional examples of suitable ACE2 activators or ACE2 agonists are disclosed, for example, in WO 2004/000365 and U.S. Pat. No. 6,194,556, the contents of each of which are incorporated herein by reference.

### Therapeutic Applications

In some embodiments, the present invention provides methods of using angiotensin-(1-7) peptides or functional equivalents, analogs or derivatives, angiotensin-(1-7) receptor agonists, ACE2 and/or ACE2 activators for treatment of peripheral vascular disease (PVD) and related diseases, disorders and conditions. Without wishing to be bound by any particular theory or hypothesis, it is contemplated that angiotensin-(1-7) peptides or functional equivalents, analogs or derivatives, angiotensin-(1-7) receptor agonists, ACE2 and/or ACE2 activators may improve blood flow and functional recovery within a target tissue by stimulating therapeutic angiogenesis.

In certain embodiments, methods and compositions of the present invention are used to stimulate repair of tissues and/or cells that are damaged by ischemia caused from a peripheral vascular disease, disorder or condition. In some embodiments, methods and compositions of the present invention are used to stimulate repair of damaged tissue in an acute condition resulting from ischemia, such as ischemic stroke. By way of non-limiting example, methods and compositions of the present invention may be used to treat peripheral vascular diseases, such as peripheral artery disease (PAD), in particular, critical limb ischemia (CLI). As used herein, the term “critical limb ischemia” or “CLI” generally refers to a condition characterized by restriction in blood or oxygen supply to the extremities (e.g., hands, feet, legs) of an individual that may result in damage or dysfunction of a tissue in the extremities. Critical limb ischemia may be caused by any of a variety of factors, such as peripheral artery disease (PAD), and may cause severe pain, skin ulcers, or sores, among other symptoms, and in some cases leads to amputation. Critical limb ischemia may be characterized by vasoconstriction, thrombosis, or embolism in one or more extremities. Any tissue in an extremity that normally receives a blood supply can experience critical limb ischemia.

In some embodiments, methods and compositions of the present invention are used to treat diabetic vascular diseases. As used herein, the term “diabetic vascular disease” refers to diseases, disorders or conditions associated with the development of blockages in the blood vessels, in particular, arteries because of diabetes. Diabetic vascular disease can be developed throughout the body. In some embodiments, diabetic vascular disease, as used herein, is developed in one or more tissues outside the heart and brain. In some embodiments, methods and compositions of the present invention are used to treat particular type of diabetic vascular diseases such as nephropathy (a kidney disease), and/or neuropathy (a condition of the nerves themselves that causes a loss of protective sensation in the toes or feet). Exemplary symptoms of diabetic vascular disease may include, but not be limited to, blurry vision, swelling of face or limbs or unexpected weight gain, foot sores, loss of feeling or a burning feeling in hands or feet, pain in legs when walking, and high blood pressure. A patient suffering from a diabetic vascular disease may eventually develop dead tissue, which is known as gangrene. It can lead to infection and ultimately to amputation.

### Peripheral Vascular Disease

Among other things, methods and compositions of the present invention are used to treat or ameliorate peripheral vascular disease. As used herein, the term “peripheral vascular disease” or “PVD” refers to a disease of the blood vessels located outside the heart and the brain.

In some embodiments, treatment refers to partial or complete alleviation, amelioration, relief, inhibition, delaying onset, reducing severity and/or incidence of peripheral artery disease in a subject. As used herein, the term, “peripheral artery disease” or “PAD” refers to a form of PVD in which there are partial or total blockage of arteries that provide blood supply to internal organs and/or limbs. In some embodiments, treatment refers to partial or complete alleviation, amelioration, relief, inhibition, delaying onset, reducing severity and/or incidence of critical limb ischemia in a subject. As used herein, the term “critical limb ischemia” or “CLI” generally refers to a condition characterized by restriction in blood or oxygen supply to the extremities (e.g., hands, arms, feet, legs) of an individual that may result in damage or dysfunction of a tissue in the extremities. Critical limb ischemia may be caused by any of a variety of factors, such as peripheral artery disease (PAD), and may cause severe pain, skin ulcers, or sores, and in some cases leads to amputation. Critical limb ischemia may be characterized by vasoconstriction, thrombosis, or embolism in one or more extremities. Any tissue in an extremity that normally receives a blood supply can experience critical limb ischemia.

In some embodiments, treatment refers to improved blood flow in a subject suffering from a peripheral vascular disease, disorder or condition. It will be appreciated that blood flow can be measured using any available methods and/or instrumentation. For example, in some embodiments, blood flow is measured using a laser Doppler. It will be appreciated that blood flow can be measured at any appropriate time before and/or after treatment. For example, in some embodiments, blood flow is measured at one or more of day 0, day 1, day 2, day 3, day 4, day 5, day 6, day 7, day 8, day 9, day 10, day 11, day 12, day 13, day 14, day 21, day 28, day 35, day 42, or day 49 of treatment. In some embodiments, blood flow measurements are expressed as a ratio of blood flow in the diseased and/or damaged tissue compared to that in a normal tissue. In some embodiments, blood flow in a diseased and/or damaged tissue is more than 20%, more than 30%, more than 40%, more than 50%, more than 60%, or more than 65% as compared to a normal tissue in the same individual. In some embodiments, blood flow in the diseased and/or damaged tissue is increased by, on average, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more per week.

In some embodiments, treatment refers to reduced or prevented necrosis (e.g., increased ischemic score) in diseased and/or damaged tissue. For example, in some embodiments, necrosis is determined by macroscopic evaluation of ischemic severity in a diseased and/or damaged tissue. It will be appreciated that necrosis can be determined by any appropriate method. For example, in some embodiments, morphological grades for necrotic areas are assigned, such as those disclosed in Goto et al. (Tokai J Exp Clin Med, 31(3):128, 2006). Exemplary morphological grades for necrotic area in mice are shown in Table 2 below.

TABLE 2

| Grade | Description |
|---|---|
| 0 | Absence of necrosis |
| 1 | Necrosis limiting to toes (toes loss) |
| 2 | Necrosis extending to a dorsum pedis (foot loss) |
| 3 | Necrosis extending to a crus (knee loss) |
| 4 | Necrosis extending to a thigh (total hind limb loss) |

In some embodiments, morphological grades for necrotic areas are decreased by about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 or more grades. In some embodiments, morphological grades for necrotic areas are decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more.

In some embodiments, treatment refers to improved limb function. It will be appreciated that limb function can be measured using any appropriate methods and/or instrumentation. For example, in some embodiments, limb function is determined by a semi-quantitative assessment of impaired use of an ischemic limb (see, e.g., Stabile, et al. Circulation 108(2):205, 2003). An exemplary assessment scale of limb function in mice are provided in Table 3 below. It will be appreciated that assessment of limb function in humans correlates with that of mice.

TABLE 3

| Grade | Description |
|---|---|
| 0 | Flexing the toes to resist gentle traction of the tail |
| 1 | Plantar flexion |
| 2 | No dragging but no planar flexion |
| 3 | Dragging of foot |

In some embodiments, grades for limb function necrotic areas are decreased by about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0 or more grades. In some embodiments, grades for limb function are decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more.

### Pharmaceutical Compositions

The pharmaceutical compositions can be in a variety of forms including oral dosage forms, topic creams, topical patches, iontophoresis forms, suppository, nasal spray and inhaler, eye drops, intraocular injection forms, depot forms, as well as injectable and infusible solutions. Methods for preparing pharmaceutical composition are well known in the art.

Pharmaceutical compositions typically contain the active agent described herein (e.g. angiotensin-(1-7) peptides or functional equivalents, analogs or derivatives, angiotensin-(1-7) receptor agonists, ACE2 and/or ACE2 activators) in an amount effective to achieve the desired therapeutic effect while avoiding or minimizing adverse side effects. Pharmaceutically acceptable preparations and salts of the active agent are provided herein and are well known in the art. For the administration of polypeptides and the like, the amount administered desirably is chosen that is therapeutically effective with few to no adverse side effects. The amount of the therapeutic or pharmaceutical composition which is effective in the treatment of a particular disease, disorder or condition depends on the nature and severity of the disease, the target site of action, the subject's weight, special diets being followed by the subject, concurrent medications being used, the administration route and other factors that are recognized by those skilled in the art. The dosage can be adapted by the clinician in accordance with conventional factors such as the extent of the disease and different parameters from the subject. Effective doses may be extrapolated from dose-response curves derived from in vitro or animal model test systems (e.g., as described by the U.S. Department of Health and Human Services, Food and Drug Administration, and Center for Drug Evaluation and Research in “Guidance for Industry: Estimating Maximum Safe Starting Dose in Initial Clinical Trials for Therapeutics in Adult Healthy Volunteers”, Pharmacology and Toxicology, July 2005, the entire contents of which are incorporated herein by reference).

Various delivery systems are known and can be used to administer active agent described herein (e.g. angiotensin-(1-7) peptides or functional equivalents, analogs or derivatives, angiotensin-(1-7) receptor agonists, ACE2 and/or ACE2 activators) or a pharmaceutical composition comprising the same. The pharmaceutical compositions described herein can be administered by any suitable route including, intravenous or intramuscular injection, intraventricular or intrathecal injection (for central nervous system administration), orally, topically, subcutaneously, intrapulmonary (e.g., inhalation), subconjunctivally, intraocularly, or via intranasal, intradermal, sublingual, vaginal, rectal or epidural routes.

Other delivery systems well known in the art can be used for delivery of the pharmaceutical compositions described herein, for example via aqueous solutions, encapsulation in microparticules, or microcapsules. The pharmaceutical compositions of the present invention can also be delivered in a controlled release system. For example, a polymeric material can be used (see, e.g., Smolen and Ball, Controlled Drug Bioavailability, Drug product design and performance, 1984, John Wiley & Sons; Ranade and Hollinger, Drug Delivery Systems, pharmacology and toxicology series, 2003, 2^{nd} edition, CRRC Press). Alternatively, a pump may be used (Saudek et al., N. Engl. J. Med. 321:574 (1989)). The compositions described herein may also be coupled to a class of biodegradable polymers useful in achieving controlled release of the drug, for example, polylactic acid, polyorthoesters, cross-linked amphipathic block copolymers and hydrogels, polyhydroxy butyric acid, and polydihydropyrans.

As described above, pharmaceutical compositions desirably include a pharmaceutically acceptable carrier. The term carrier refers to diluents, adjuvants, excipients or vehicles with which the peptide, peptide derivative or peptidomimetic is administered. Such pharmaceutical carriers include sterile liquids such as water and oils including mineral oil, vegetable oil (e.g., soybean oil or corn oil), animal oil or oil of synthetic origin. Aqueous glycerol and dextrose solutions as well as saline solutions may also be employed as liquid carriers of the pharmaceutical compositions of the present invention. The choice of the carrier depends on factors well recognized in the art, such as the nature of the peptide, peptide derivative or peptidomimetic, its solubility and other physiological properties as well as the target site of delivery and application. Examples of suitable pharmaceutical carriers are described in Remington: The Science and Practice of Pharmacy by Alfonso R. Gennaro, 2003, 21^{th} edition, Mack Publishing Company. Moreover, suitable carriers for oral administration are known in the art and are described, for example, in U.S. Pat. Nos. 6,086,918, 6,673,574, 6,960,355, and 7,351,741 and in WO2007/131286, the disclosures of which are hereby incorporated by reference.

Further pharmaceutically suitable materials that may be incorporated in pharmaceutical preparations include absorption enhancers including those intended to increase paracellular absorption, pH regulators and buffers, osmolarity adjusters, preservatives, stabilizers, antioxidants, surfactants, thickeners, emollient, dispersing agents, flavoring agents, coloring agents, and wetting agents.

Examples of suitable pharmaceutical excipients include, water, glucose, sucrose, lactose, glycol, ethanol, glycerol monostearate, gelatin, starch flour (e.g., rice flour), chalk, sodium stearate, malt, sodium chloride, and the like. The pharmaceutical compositions comprising Angiotensin polypeptides can take the form of solutions, capsules, tablets, creams, gels, powders sustained release formulations and the like. The composition can be formulated as a suppository, with traditional binders and carriers such as triglycerides (see Remington: The Science and Practice of Pharmacy by Alfonso R. Gennaro, 2003, 21^{th} edition, Mack Publishing Company). Such compositions contain a therapeutically effective amount of the therapeutic composition, together with a suitable amount of carrier so as to provide the form for proper administration to the subject. The formulations are designed to suit the mode of administration and the target site of action (e.g., a particular organ or cell type).

The pharmaceutical compositions comprising the active agent described herein (e.g. angiotensin-(1-7) peptides or functional equivalents, analogs or derivatives, angiotensin-(1-7) receptor agonists, ACE2 and/or ACE2 activators) also include compositions formulated as neutral or salt forms. Pharmaceutically acceptable salts include those that form with free amino groups and those that react with free carboxyl groups. Non-toxic alkali metal, alkaline earth metal, and ammonium salts commonly used in the pharmaceutical industry include sodium, potassium, lithium, calcium, magnesium, barium, ammonium, and protamine zinc salts, which are prepared by methods well known in the art. Also included are non-toxic acid addition salts, which are generally prepared by reacting the compounds of the present invention with suitable organic or inorganic acid. Representative salts include the hydrobromide, hydrochloride, valerate, oxalate, oleate, laureate, borate, benzoate, sulfate, bisulfate, acetate, phosphate, tysolate, citrate, maleate, fumarate, tartrate, succinate, napsylate salts, and the like.

Examples of fillers or binders that may be used in accordance with the present invention include acacia, alginic acid, calcium phosphate (dibasic), carboxymethylcellulose, carboxymethylcellulose sodium, hydroxyethylcellulose, hydroxypropylcellulose, hydroxypropylmethylcellulose, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. In certain embodiments, a filler or binder is microcrystalline cellulose.

Examples of disintegrating agents that may be used include alginic acid, carboxymethylcellulose, carboxymethylcellulose sodium, hydroxypropylcellulose (low substituted), microcrystalline cellulose, powdered cellulose, colloidal silicon dioxide, sodium croscarmellose, crospovidone, methylcellulose, polacrilin potassium, povidone, sodium alginate, sodium starch glycolate, starch, disodium disulfite, disodium edathamil, disodium edetate, disodiumethylenediaminetetraacetate (EDTA) crosslinked polyvinylpyrrolidones, pregelatinized starch, carboxymethyl starch, sodium carboxymethyl starch, microcrystalline cellulose.

Examples of lubricants include calcium stearate, canola oil, glyceryl palmitostearate, hydrogenated vegetable oil (type I), magnesium oxide, magnesium stearate, mineral oil, poloxamer, polyethylene glycol, sodium lauryl sulfate, sodium stearate fumarate, stearic acid, talc and, zinc stearate, glyceryl behapate, magnesium lauryl sulfate, boric acid, sodium benzoate, sodium acetate, sodium benzoate/sodium acetate (in combination), DL-leucine.

Examples of silica flow conditioners include colloidal silicon dioxide, magnesium aluminum silicate and guar gum. Another most preferred silica flow conditioner consists of silicon dioxide.

Examples of stabilizing agents include acacia, albumin, polyvinyl alcohol, alginic acid, bentonite, dicalcium phosphate, carboxymethylcellulose, hydroxypropylcellulose, colloidal silicon dioxide, cyclodextrins, glyceryl monostearate, hydroxypropyl methylcellulose, magnesium trisilicate, magnesium aluminum silicate, propylene glycol, propylene glycol alginate, sodium alginate, carnauba wax, xanthan gum, starch, stearate(s), stearic acid, stearic monoglyceride and stearyl alcohol.

In some embodiments, the present invention contemplates oral formulations containing the active agent described herein (e.g. angiotensin-(1-7) peptides or functional equivalents, analogs or derivatives, angiotensin-(1-7) receptor agonists, ACE2 and/or ACE2 activators). For example, pharmaceutical compositions described herein may include a cyclodextrin or cyclodextrin derivative. Cyclodextrins are generally made up of five or more α-D-glycopyranoside unites linked 1->4. Typically, cyclodextrins contain a number of glucose monomers ranging from six to eight units in a ring, creating a cone shape (α-cyclodextrin: six membered sugar ring molecule, β-cyclodextrin: seven sugar ring molecule, γ-cyclodextrin: eight sugar ring molecule). Exemplary cyclodextrins and cyclodextrin derivatives are disclosed in U.S. Pat. No. 7,723,304, U.S. Publication No. 2010/0196452, and U.S. Publication No. 2010/0144624, the entire contents of each of which are incorporated herein by reference. For example, in some embodiments, a cyclodextrin in accordance with the present invention is an alkylated cyclodextrin, hydroxyalkylated cyclodextrin, or acylated cyclodextrin. In some embodiments, a cyclodextrin is a hydroxypropyl β-cyclodextrin. Exemplary cyclodextrin derivatives are disclosed in Szejtli, J. Chem Rev, (1998), 98, 1743-1753; and Szente, L and Szejtli, J., Advance Drug Delivery Reviews, 36 (1999) 17-28, the entire contents of each of which are hereby incorporated by reference. Examples of cyclodextin derivatives include methylated cyclodextrins (e.g., RAMEB; randomly methylated β-cyclodextrin); hydroxyalkylated cyclodextrins (hydroxypropyl-β-cyclodextrin and hydroxypropyl γ-cyclodextrin); acetylated cyclodextrins (acetyl-γ-cyclodextrin); reactive cyclodextrins (chlorotriazinyl β-cyclodextrin); and branched cyclodextrins (glucosyl- and maltosyl β-cyclodextrin); acetyl-γ-cyclodextrin; acetyl-β-cyclodextrin, sulfobutyl-βcyclodextrin, sulfated α-, β- and γ-cyclodextrins; sulfoalkylated cyclodextrins; and hydroxypropyl β-cyclodextrin.

### Dosing

Typically, active agent described herein (e.g. angiotensin-(1-7) peptides or functional equivalents, analogs or derivatives, angiotensin-(1-7) receptor agonists, ACE2 and/or ACE2 activators) in an amount ranging from 0.001 to 100 mg/kg/day is administered to the subject. For example, in some embodiments, about 0.01 mg/kg/day to about 25 mg/kg/day, about 1 mg/kg/day to about 20 mg/kg/day, 0.2 mg/kg/day to about 10 mg/kg/day, about 0.02 mg/kg/day to about 0.1 mg/kg/day, or about 1 mg/kg/day to about 100 mg/kg/day is administered to the subject. In some embodiments, active agent described herein (e.g. angiotensin-(1-7) peptides or functional equivalents, analogs or derivatives, angiotensin-(1-7) receptor agonists, ACE2 and/or ACE2 activators) in an amount of about 10 μg/kg/day, 50 μg/kg/day, 100 μg/kg/day, 200 μg/kg/day, 300 μg/kg/day, 400 μg/kg/day, 500 μg/kg/day, 600 μg/kg/day, 700 μg/kg/day, 800 μg/kg/day, 900 μg/kg/day, or 1000 μg/kg/day is administered to the subject.

In some embodiments, the angiotensin (1-7) peptide is administered at an effective dose ranging from about 1-1,000 μg/kg/day (e.g., ranging from about 1-900 μg/kg/day, 1-800 μg/kg/day, 1-700 μg/kg/day, 1-600 μg/kg/day, 1-500 μg/kg/day, 1-400 μg/kg/day, 1-300 μg/kg/day, 1-200 μg/kg/day, 1-100 μg/kg/day, 1-90 μg/kg/day, 1-80 μg/kg/day, 1-70 μg/kg/day, 1-60 μg/kg/day, 1-50 μg/kg/day, 1-40 μg/kg/day, 1-30 μg/kg/day, 1-20 μg/kg/day, 1-10 μg/kg/day). In some embodiments, the angiotensin (1-7) peptide is administered at an effective dose ranging from about 1-500 μg/kg/day. In some embodiments, the angiotensin (1-7) peptide is administered at an effective dose ranging from about 1-100 μg/kg/day. In some embodiments, the angiotensin (1-7) peptide is administered at an effective dose ranging from about 1-60 μg/kg/day. In some embodiments, the angiotensin (1-7) peptide is administered at an effective dose selected from about 1, 2, 4, 6, 8, 10, 15, 20, 25, 30, 35, 40, 45, 50, 75, 100, 150, 200, 250, 300, 350, 400, 450, 500, 550, 600, 650, 700, 750, 800, 850, 900, 950, or 1,000 ug/kg/day.

In some embodiments, a therapeutically effective amount of an angiotensin-(1-7) peptide or functional equivalent, analog or derivative, angiotensin-(1-7) receptor agonist, ACE2 and/or ACE2 activator) may be an amount ranging from about 10-1,000 mg (e.g., about 20 mg-1,000 mg, 30 mg-1,000 mg, 40 mg-1,000 mg, 50 mg-1,000 mg, 60 mg-1,000 mg, 70 mg-1,000 mg, 80 mg-1,000 mg, 90 mg-1,000 mg, about 10-900 mg, 10-800 mg, 10-700 mg, 10-600 mg, 10-500 mg, 100-1000 mg, 100-900 mg, 100-800 mg, 100-700 mg, 100-600 mg, 100-500 mg, 100-400 mg, 100-300 mg, 200-1000 mg, 200-900 mg, 200-800 mg, 200-700 mg, 200-600 mg, 200-500 mg, 200-400 mg, 300-1000 mg, 300-900 mg, 300-800 mg, 300-700 mg, 300-600 mg, 300-500 mg, 400 mg-1,000 mg, 500 mg-1,000 mg, 100 mg-900 mg, 200 mg-800 mg, 300 mg-700 mg, 400 mg-700 mg, and 500 mg-600 mg). In some embodiments, an angiotensin (1-7) peptide or angiotensin (1-7) receptor agonist is present in an amount of or greater than about 10 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg. In some embodiments, an angiotensin (1-7) peptide or angiotensin (1-7) receptor agonist is present in an amount of or less than about 1000 mg, 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, or 100 mg. In some embodiments, the therapeutically effective amount described herein is provided in one dose. In some embodiments, the therapeutically effective amount described herein is provided in one day.

In other embodiments, a therapeutically effective amount may be, for example, about 0.001 mg/kg weight to 500 mg/kg weight, e.g., from about 0.001 mg/kg weight to 400 mg/kg weight, from about 0.001 mg/kg weight to 300 mg/kg weight, from about 0.001 mg/kg weight to 200 mg/kg weight, from about 0.001 mg/kg weight to 100 mg/kg weight, from about 0.001 mg/kg weight to 90 mg/kg weight, from about 0.001 mg/kg weight to 80 mg/kg weight, from about 0.001 mg/kg weight to 70 mg/kg weight, from about 0.001 mg/kg weight to 60 mg/kg weight, from about 0.001 mg/kg weight to 50 mg/kg weight, from about 0.001 mg/kg weight to 40 mg/kg weight, from about 0.001 mg/kg weight to 30 mg/kg weight, from about 0.001 mg/kg weight to 25 mg/kg weight, from about 0.001 mg/kg weight to 20 mg/kg weight, from about 0.001 mg/kg weight to 15 mg/kg weight, from about 0.001 mg/kg weight to 10 mg/kg weight. In some embodiments, the therapeutically effective amount described herein is provided in one dose. In some embodiments, the therapeutically effective amount described herein is provided in one day.

In still other embodiments, a therapeutically effective amount may be, for example, about 0.0001 mg/kg weight to 0.1 mg/kg weight, e.g. from about 0.0001 mg/kg weight to 0.09 mg/kg weight, from about 0.0001 mg/kg weight to 0.08 mg/kg weight, from about 0.0001 mg/kg weight to 0.07 mg/kg weight, from about 0.0001 mg/kg weight to 0.06 mg/kg weight, from about 0.0001 mg/kg weight to 0.05 mg/kg weight, from about 0.0001 mg/kg weight to about 0.04 mg/kg weight, from about 0.0001 mg/kg weight to 0.03 mg/kg weight, from about 0.0001 mg/kg weight to 0.02 mg/kg weight, from about 0.0001 mg/kg weight to 0.019 mg/kg weight, from about 0.0001 mg/kg weight to 0.018 mg/kg weight, from about 0.0001 mg/kg weight to 0.017 mg/kg weight, from about 0.0001 mg/kg weight to 0.016 mg/kg weight, from about 0.0001 mg/kg weight to 0.015 mg/kg weight, from about 0.0001 mg/kg weight to 0.014 mg/kg weight, from about 0.0001 mg/kg weight to 0.013 mg/kg weight, from about 0.0001 mg/kg weight to 0.012 mg/kg weight, from about 0.0001 mg/kg weight to 0.011 mg/kg weight, from about 0.0001 mg/kg weight to 0.01 mg/kg weight, from about 0.0001 mg/kg weight to 0.009 mg/kg weight, from about 0.0001 mg/kg weight to 0.008 mg/kg weight, from about 0.0001 mg/kg weight to 0.007 mg/kg weight, from about 0.0001 mg/kg weight to 0.006 mg/kg weight, from about 0.0001 mg/kg weight to 0.005 mg/kg weight, from about 0.0001 mg/kg weight to 0.004 mg/kg weight, from about 0.0001 mg/kg weight to 0.003 mg/kg weight, from about 0.0001 mg/kg weight to 0.002 mg/kg weight. In some embodiments, the therapeutically effective dose may be 0.0001 mg/kg weight, 0.0002 mg/kg weight, 0.0003 mg/kg weight, 0.0004 mg/kg weight, 0.0005 mg/kg weight, 0.0006 mg/kg weight, 0.0007 mg/kg weight, 0.0008 mg/kg weight, 0.0009 mg/kg weight, 0.001 mg/kg weight, 0.002 mg/kg weight, 0.003 mg/kg weight, 0.004 mg/kg weight, 0.005 mg/kg weight, 0.006 mg/kg weight, 0.007 mg/kg weight, 0.008 mg/kg weight, 0.009 mg/kg weight, 0.01 mg/kg weight, 0.02 mg/kg weight, 0.03 mg/kg weight, 0.04 mg/kg weight, 0.05 mg/kg weight, 0.06 mg/kg weight, 0.07 mg/kg weight, 0.08 mg/kg weight, 0.09 mg/kg weight, or 0.1 mg/kg weight. The effective dose for a particular individual can be varied (e.g., increased or decreased) over time, depending on the needs of the individual. In some embodiments, the therapeutically effective amount described herein is provided in one dose. In some embodiments, the therapeutically effective amount described herein is provided in one day.

### V. Kits

In certain embodiments, kits or other articles of manufacture are provided which comprise the active agent described herein (e.g. angiotensin-(1-7) peptides or functional equivalents, analogs or derivatives, angiotensin-(1-7) receptor agonists, ACE2 and/or ACE2 activators), tools for administration, and/or instructions for use. For example, kits or other articles of manufacture may include a container, a catheter and any other articles, devices or equipment useful in administration. Suitable containers include, for example, bottles, vials, syringes (e.g., pre-filled syringes), ampules, cartridges, reservoirs, or lyo-jects. The container may be formed from a variety of materials such as glass or plastic. In certain embodiments, a container is a pre-filled syringe. Suitable pre-filled syringes include, but are not limited to, borosilicate glass syringes with baked silicone coating, borosilicate glass syringes with sprayed silicone, or plastic resin syringes without silicone.

Typically, the container holds formulations containing the active agent described herein (e.g. angiotensin-(1-7) peptides or functional equivalents, analogs or derivatives, angiotensin-(1-7) receptor agonists, ACE2 and/or ACE2 activators) and a label on, or associated with, the container that may indicate directions for reconstitution and/or use as described herein.

### EXEMPLIFICATION

### Example 1

### Angiotensin (1-7) Treatment in an Animal Model of Chronic Hind Limb Ischemia Improved Blood Flow and Limb Function

The present Example demonstrates that angiotensin (1-7) can be used to effectively treat ischemic diseases. In this example, a linear angiotensin peptide TXA127 having an amino acid sequence of Asp¹-Arg²-Val³-Tyr⁴-Ile⁵-His⁶-Pro⁷ (SEQ ID NO: 1) was used as an example to assess the therapeutic effect of angiotensin (1-7) in a mouse hind limb ischemia model.

Hind Limb Ischemia Model

A stable hind limb ischemia model has been described previously and is generally characterized by uniform ischemic damage useful for examining the effect of various therapies (Goto, et al. Tokai J Exp Clin Med, 31(3):128 2006; Kang Y, et al. PLoS One. 2009; 4(1):e4275)). The hind limb ischemia model in mice used in this example involves two ligations of the proximal end of the femoral artery and its dissection between the two ligatures. The surgery causes obstruction of the blood flow and subsequently leads to severe ischemic damage (Goto, et al.; Kang, et al.). In this experiment, healthy adult female Balb/c mice were used. Hind limb ischemia was induced in mice using protocols previously described. Briefly, Balb/c female mice were maintained on a standard diet with water available ad libitum. Mice were anesthetized and an incision was made in the skin in the inguinal area. The femoral artery was ligated twice with 6-0 silk thread and transected between the ligatures after this the wound was closed with 4-0 silk thread and the mouse was allowed to recover.

Administration of TXA 127

An Angiotensin (1-7) polypeptide composition (TXA127) and vehicle control (DPBS) were supplied as ready to use solutions and were stored at 4° C. until use. TXA127 was injected subcutaneously (500 μg/kg) daily starting on day 1, 24 hours after inducing ischemia, until the end of the study. Negative control mice were injected subcutaneously with a vehicle. Table 4 provides animal group allocation.

TABLE 4
Group allocation

| | Surgical | Treatment | Dose | | Route of |
|---|---|---|---|---|---|
| Group | Procedure | (Lot) | mg/kg | Volume | Administration |
| 1F | √ | Negative | NA | 5 ml/kg | SC |
| (N = 26) | | control | | | |
| | | (vehicle) | | | |
| 2F | √ | Angiotensin | 500 μg/kg | 5 ml/kg | |
| (N = 24) | | (TXA127) | | | SC |

Evaluation of Ischemia

Body Weight

Body weight of animals was measured before the surgery and once weekly thereafter.

Blood Flow

Blood flow in legs from both sides of the animals was measured with a non contact laser Doppler before surgery and on days: 1, 7, 15, 21, 28, 35, 42 and 49 post operation. Blood flow measurements were expressed as the ratio of the flow in the ischemic limb to that in the normal limb.

Macroscopic Assessment of Ischemic Severity

Macroscopic evaluation of the ischemic limb was done once a week post operation by using morphological grades for necrotic area (Goto, et al. Tokai J Exp Clin Med, 31(3):128 2006) as shown in Table 5.

TABLE 5
Morphological grades for necrotic area

| Grade | Description |
|---|---|
| 0 | absence of necrosis |
| 1 | necrosis limiting to toes (toes loss), |
| 2 | necrosis extending to a dorsum pedis (foot loss), |
| 3 | necrosis extending to a crus (knee loss) |
| 4 | necrosis extending to a thigh (total hind-limb loss) |

In Vivo Assessment of Limb Function and Ischemic Damage

Semi-quantitative assessment of impaired use of the ischemic limb was performed once a week post-surgery using the scale shown in Table 6 (Stabile et al, Circulation 108(2):205 2003).

TABLE 6
Assessment of limb function

| Grade | Description |
|---|---|
| 0 | flexing the toes to resist gentle traction of the tail |
| 1 | plantar flexion |
| 2 | no dragging but no plantar flexion |
| 3 | dragging of foot |

Limb function is graded as “Not applicable” in case of partial or full limb amputation. In such case blood flow measurements will not be included in the statistical analysis.

Tissue Fixation

On the day when animals were sacrificed, the quadriceps muscles of ischemic and control legs were removed and fixed in 4% buffered formalin for analysis.

Results

A stable severe ischemia model generated using the method described herein was used to assess TXA 127 angiogenesis efficacy after repeated subcutaneous administration.

Body Weight

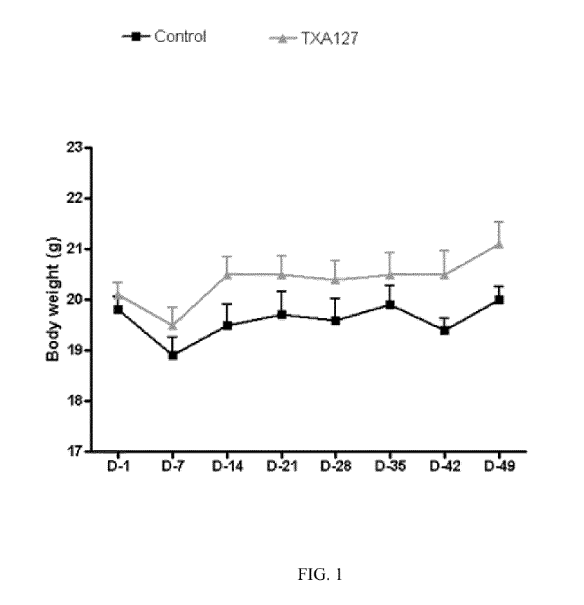

Exemplary body weight distribution is summarized in FIG. 1. Throughout the study, no statistically significant differences in body weight of the animals were observed.

Blood Flow

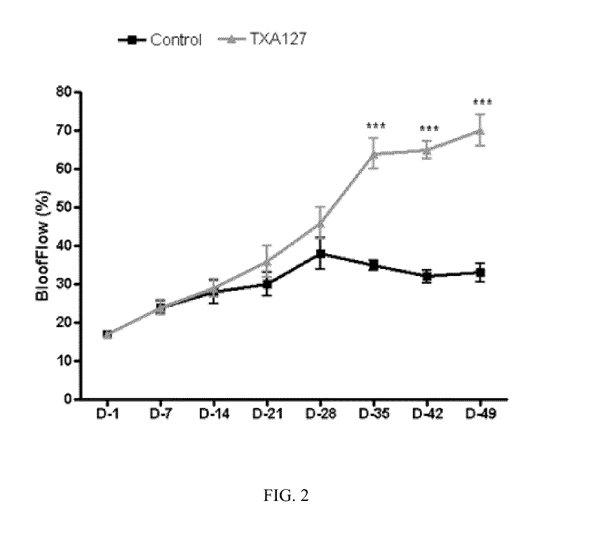

From day 35 up to the termination of the study on day 49, statistically significant improvement in blood flow was observed in the TXA 127 treated animal group (2F) as compared to the vehicle (control) treated animal group (1F). Exemplary results are summarized in FIG. 2.

Statistical analysis for FIG. 2 was carried out using two-way ANOVA for repeated measures, followed by Bonferroni post hoc tests. Comparison of control group 1F to TXA 127 treated group 2F showed statistically significant difference on day 35 (p<0.001).

Assessment of Ischemic Severity In Vivo

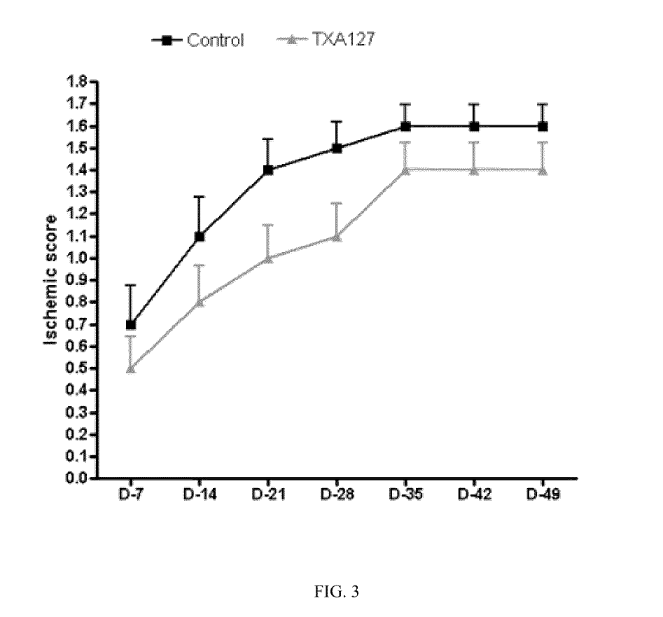

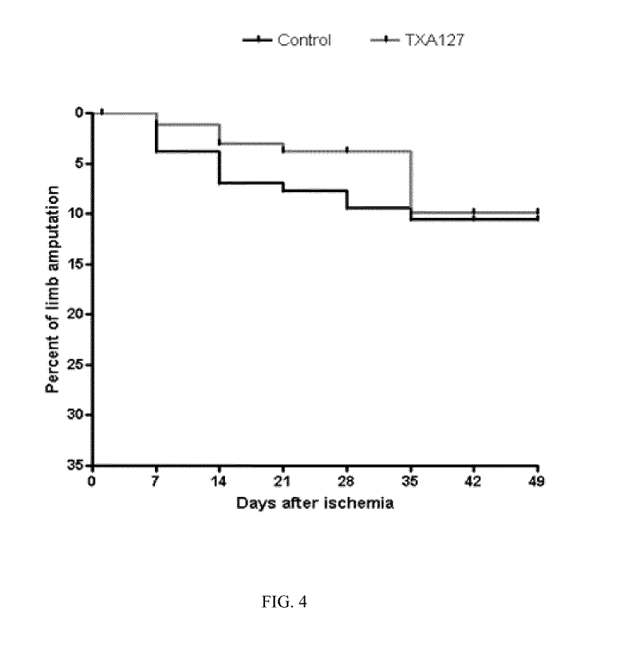

Using graded morphological scales for necrotic area, the ischemic limb was evaluated on day 7 and day 49. Limb amputation was found in both group of animals—in the control group 1F it was 60% and in the TXA 127 treated group 2F it was 48%. Ischemic severity was also different in the control and TXA 127 treated groups. A trend evident to decreasing severity was seen in TXA 127 treated group compared to control treated group. These results are summarized in FIG. 3. Moreover, most limb amputation in the TXA 127 treated group occurs only on day 35 after induction of hind limb ischemia as shown in FIG. 4 (where 0% represents no amputation and the decrease reflects the increase in amputations throughout the study).

Assessment of Limb Function In Vivo

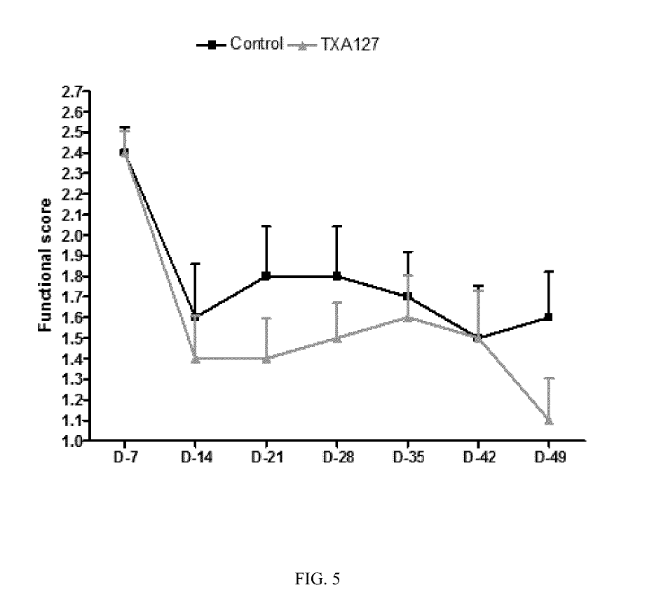

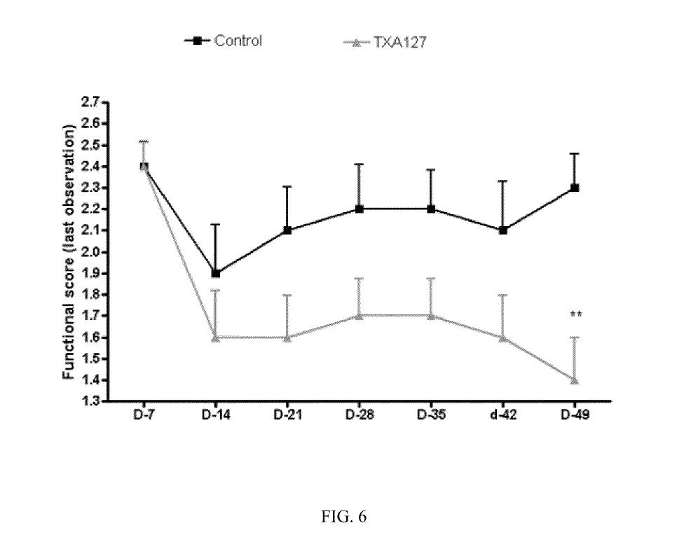

Semi-quantitative assessment of impaired use of the ischemic limb was performed from day 7 up to day 49 by using graded functional scales. An improvement in limb function was found in the TXA127 treated group (2f) as compared to the vehicle or control treated group (1F) of animals up to day 49 after induction of hind limb ischemia. The differences however, were not statistically significant. These results are summarized in FIG. 5. This trend reached statistical significance when “last measure carried forward” method of analysis was employed as shown in FIG. 6.

Statistical analysis for FIG. 6 using the “last measure carried forward” method employed using the two-way ANOVA for repeated measures, followed by Bonferroni post hoc tests. Comparison of control group 1F to TXA 127 treated group 2F showed statistically significant difference on day 49 (p<0.01).

Taken together, these results demonstrate that TXA127 can effectively treat ischemic diseases by stimulating blood flow and tissue repair. For example, it has been found that subcutaneous administration of TXA127 restored blood flow to 71% of its normal values. Blood flow perfusion restoration is consistent with other findings showing that TXA127 treatment improves limb function and decreases ischemic amuptations. Furthermore, TXA127 treatment also alleviates damage to limbs that have undergone ischemic stress. These findings indicate that angiotensin (1-7) can be used for therapeutic angiogenesis to treat various ischmeic diseases such as critical limb ischemia and other peripheral vascular diseases.

### Example 2

### PanCyte Treatment in an Animal Model of Chronic Hind Limb Ischemia Improved Blood Flow and Limb Function

The present Example demonstrates that PanCyte can be used to effectively treat ischemic diseases. In this example, a cyclic angiotensin peptide having an amino acid sequence of Asp¹-Arg²-Val³-Ser⁴-Ile⁵-His⁶-Cys⁷ (SEQ ID NO:22) was used as an example to assess the therapeutic effect of PanCyte in a mouse hind limb ischemia model.

A total of 49 female mice were utilized, divided into three groups: 16 in group 1F, 17 in group 2F and 16 in group 3F. The number of the groups and the total number of animals was based on previous studies demonstrating that this was the minimum number of animals per group sufficient to obtain indicative/significant information. Table 7 shows the design of each group.

TABLE 7
Group Design

| | Surgical | Treatment | Dose | | Route of |
|---|---|---|---|---|---|
| Group | Procedure | (Lot) | mg/kg | Volume | Administration |
| 1F | √ | Negative | NA | 5 ml/kg | SC |
| (N = 16) | | control | | | |
| | | (vehicle) | | | |
| 2F | | PanCyte | 500 μg/kg | 5 ml/kg | SC |
| (N = 17) | | | | | |
| 3F | √ | PanCyte | 50 μg/kg | 5 ml/kg | SC |
| (N = 16) | | | | | |

Hind Limb Ischemia Model

The model used in this example is the same as for Example 1. Briefly, the hind limb ischemia model in mice used in this example involves two ligations of the proximal end of the femoral artery and its dissection between the two ligatures. The surgery causes obstruction of the blood flow and subsequently leads to severe ischemic damage (Goto, et al.; Kang, et al.). In this experiment, healthy adult female Balb/c mice were used. Hind limb ischemia was induced in mice using protocols previously described. Briefly, Balb/c female mice were maintained on a standard diet with water available ad libitum. Mice were anesthetized and an incision was made in the skin in the inguinal area. The femoral artery was ligated twice with 6-0 silk thread and transected between the ligatures after this the wound was closed with 4-0 silk thread and the mouse was allowed to recover.

Administration of PanCyte

An Angiotensin (1-7) polypeptide composition (PanCyte) and vehicle control (DPBS) were supplied as ready to use solutions and were stored at 4° C. until use. PanCyte was injected subcutaneously (500 μg/kg or 50 μg/kg) daily starting on day 1, 24 hours after inducing ischemia, until the end of the study. Negative control mice were injected subcutaneously with a vehicle.

Evaluation of Ischemia

Body Weight

Body weight of animals was measured before the surgery and once weekly thereafter.

Blood Flow

Blood flow in legs from both sides of the animals was measured with a non contact laser Doppler before surgery and on days: 1, 7, 15, 21, 28, 35, 42 and 49 post operation. Blood flow measurements were expressed as the ratio of the flow in the ischemic limb to that in the normal limb.

Macroscopic Assessment of Ischemic Severity

Macroscopic evaluation of the ischemic limb was done once a week post operation by using morphological grades for necrotic area as shown in Table 5 above.

In Vivo Assessment of Limb Function and Ischemic Damage

Semi-quantitative assessment of impaired use of the ischemic limb was performed once a week post-surgery using the scale shown in Table 6 above.

Limb function was graded as “Not applicable” in case of partial or full limb amputation. In such case blood flow measurements will not be included in the statistical analysis.

Tissue Fixation

On the day when animals were sacrificed, the quadriceps muscles of ischemic and control legs were removed and fixed in 2.5% buffered paraformaldehyde or zinc fixative for analysis.

Results

A stable severe ischemia model generated using the method described herein was used to assess PanCyte angiogenesis efficacy after repeated subcutaneous administration.

Body Weight

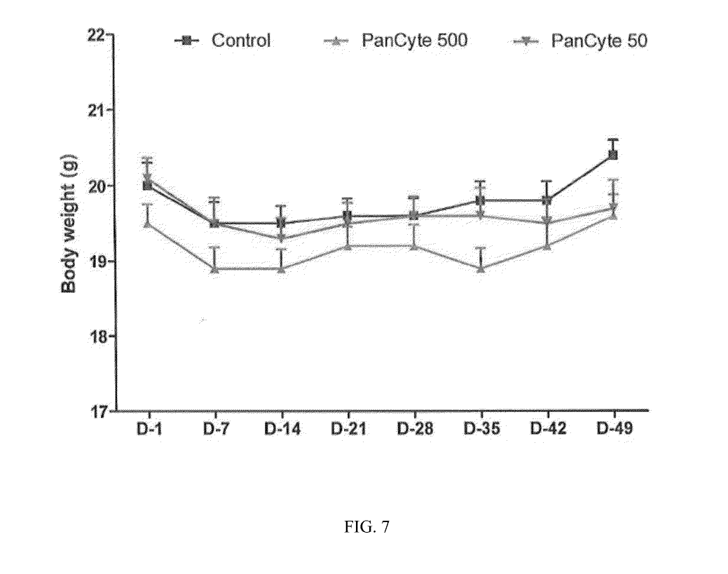

Exemplary body weights are shown in FIG. 7. Throughout the study, no statistically significant differences in body weight of the animals were observed.

Blood Flow

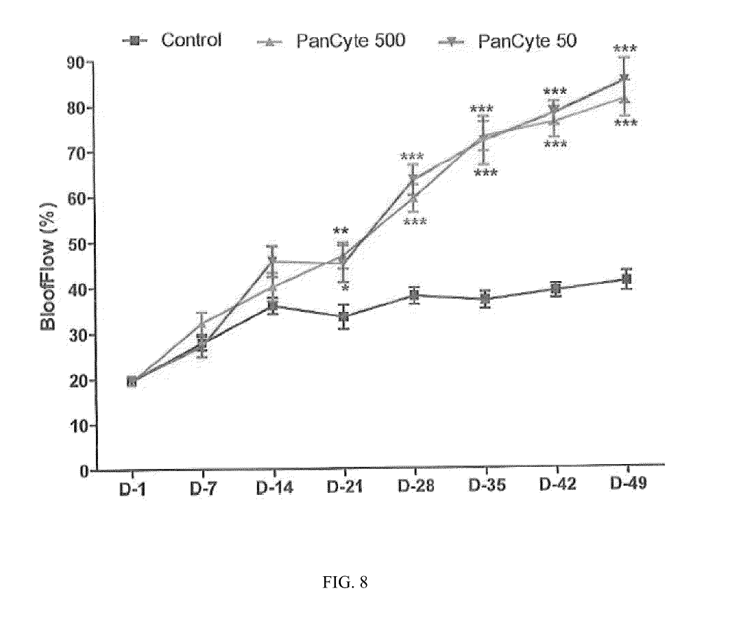

From day 21 up to study termination on day 49, statistically significant improvement in blood flow was observed in the animal groups treated with PanCyte (2F and 3F), compared to vehicle treated control (1 F). (FIG. 8).

Statistical analysis of the data shown in FIG. 8 was carried out using two-way ANOVA for repeated measures, followed by Bonferroni post hoc tests. Comparison of control group 1F to PanCyte treated groups 2F and 3F showed statistically significant differences from day 21 up to day 49 (p<0.05-0.001).

### Assessment of Limb Function

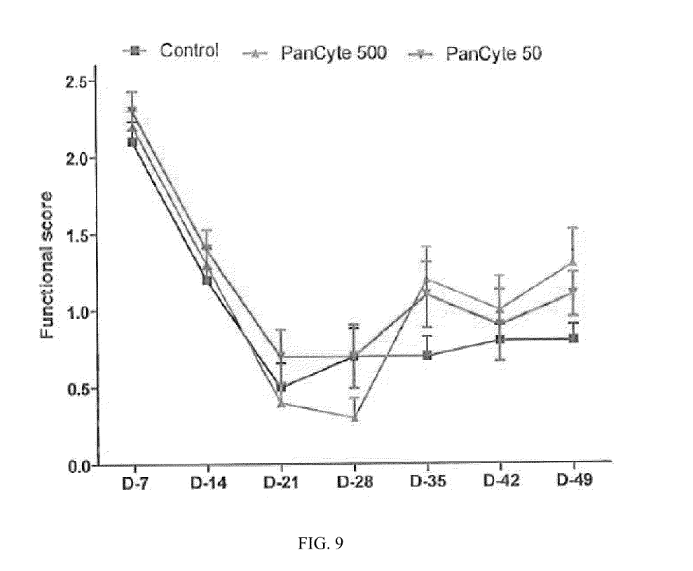

Semi-quantitative assessment of impaired use of the ischemic limb was performed on days 7 up to 49 by using graded functional scales. An improvement in limb function was observed in group treated with PanCyte 500 μg/kg 2F versus control group 1F of animals on day 28 after hindlimb ischemia (see FIG. 9).

Capillary Density

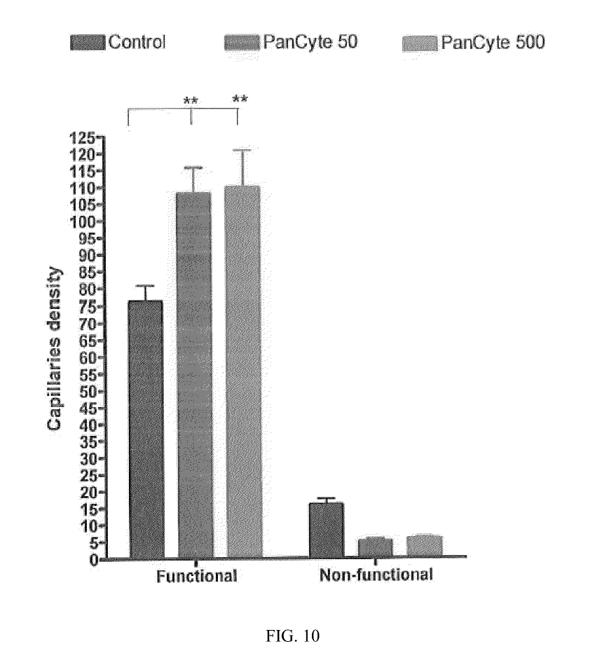

Sections of muscle samples were taken from the same areas in 6-7 animals from each group. Capillaries were counted under a microscope in a total 12 random fields from different sections. Density was expressed as the mean number of capillaries per field of view. Treatment with PanCyle significantly increased the number of capillaries 49 days after the treatment beginning. This effect was found in both treated groups of animals (FIG. 10).

This example shows that SC administration of PanCyte at either 50 μg/kg or 500 μg/kg may restore blood flow to 85% of its normal values after an ischemic event. Further, histological analysis revealed an increase in the capillary density in both animals groups treated with PanCyte compare to the control group. Taken together, these results demonstrate that PanCyte can effectively treat ischemic diseases by stimulating blood flow and tissue repair. These findings indicate that PanCyte can be used for therapeutic angiogenesis to treat various ischmeic diseases such as critical limb ischemia and other peripheral vascular diseases.

### Example 3

### Lower Dose PanCyte and Continuous Infusion Treatments in an Animal Model of Chronic Hind Limb Ischemia Improved Blood Flow and Limb Function

The present Example demonstrates that doses of PanCyte between 1 μg/kg and 50 μg/kg can be used to effectively treat ischemic diseases. In this example, a cyclic angiotensin peptide having an amino acid sequence of Asp¹-Arg²-Val³-Ser⁴-Ile⁵-His⁶-Cys (SEQ ID NO:22) was used to assess the therapeutic effect of PanCyte in a mouse hind limb ischemia model.

A total of 98 female mice were utilized, divided into three groups: 15 in group 1F, 17 in group 2F, 17 in group 3F, 16 in group 4F, 17 in group 5F, and 16 in group 6F. The number of the groups and the total number of animals was based on previous studies demonstrating that this was the minimum number of animals per group sufficient to obtain indicative/significant information. Table 8 shows the design of each group.

TABLE 8
Group Design

| | Surgical | | Dose | | Route of |
|---|---|---|---|---|---|
| Group | Procedure | Treatment | mg/kg | Volume | Administration |
| 1F | √ | Negative | NA | 5 ml/kg | SC |
| (N = 15) | | control | | | |
| | | (vehicle) | | | |
| 2F | √ | PanCyte | 1 μg/kg | 5 ml/kg | SC |
| (N = 17) | | | | | |
| 3F | √ | PanCyte | 5 μg/kg | 5 ml/kg | SC |
| (N = 17) | | | | | |
| 4F | √ | PanCyte | 25 μg/kg | 5 ml/kg | S C |
| (N = 16) | | | | | |
| 5F | √ | PanCyte | 50 μg/kg | 5 ml/kg | SC |
| (N = 17) | | | | | |
| 6F | √ | PanCyte | 50 μg/kg | 100 μl | SC by Alzet |
| (N = 16) | | | | | pump |

Hind Limb Ischemia Model

The model and procedures used for this example is the same as for those in Examples 1 and 2, unless otherwise specified.

Administration of PanCyte

An Angiotensin (1-7) polypeptide composition (PanCyte, cyclized Asp¹-Arg²-Val³-Ser⁴-Ile⁵-His⁶-Cys (SEQ ID NO:22)) and vehicle control (DPBS) were supplied as ready to use solutions and were stored at 4° C. until use. In groups 2F-5F, PanCyte was injected subcutaneously (1 μg/kg, 5 μg/kg, 25 μg/kg, 50 μg/kg) daily starting on day 1, 24 hours after inducing ischemia, until the end of the study. In group 6F, an osmotic Alzet pump was implanted subcutaneously and provided for continuous release of PanCyte over the duration of the study. Negative control mice were injected subcutaneously with vehicle (DPBS).

Results

A stale severe ischemia model generated using the method described herein was used to assess PanCyte angiogenesis efficacy after repeated subcutaneous administration.

Body Weight

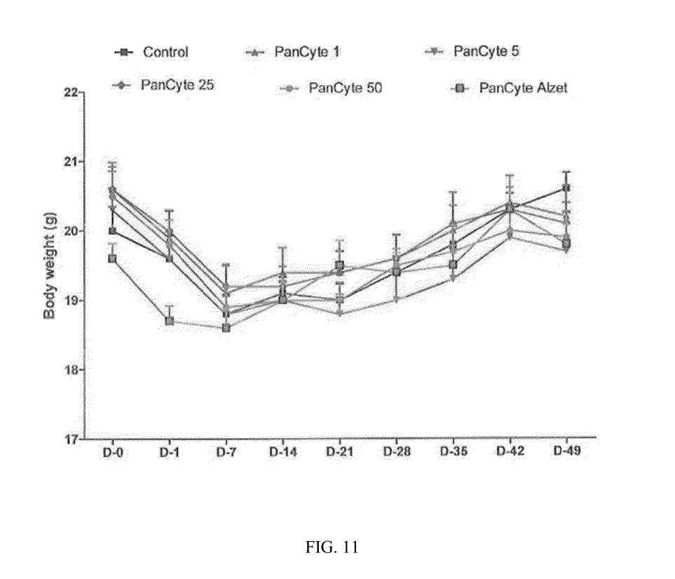

Exemplary body weights are shown in FIG. 11. Throughout the study, no statistically significant differences in body weight were observed.

Blood Flow

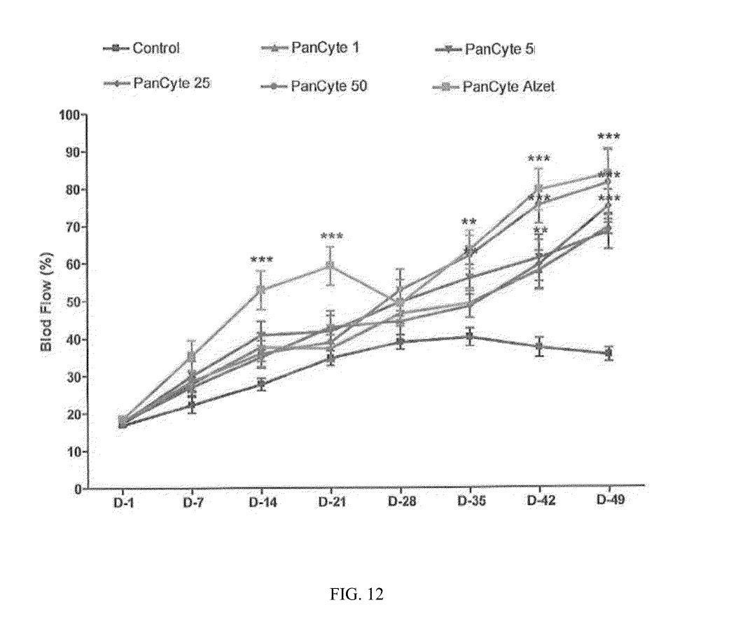

Statistically significant improvement in blood flow was observed in the animal group treated by continuous PanCyte administration using Alzet pump (6F), compared to vehicle treated control (1F), starting on day 14 of the study and up to study termination on day 49 (see FIG. 12). In group 5F treated with PanCyte in the similar dose, but given by daily injections, significant improvement in blood flow compared to control was observed from day 35 up to day 49. In the other treatment groups, blood flow improvement reached statistically significance on days 42 and day 49 (groups 2F, 3F and 4F).

Assessment of Limb Function

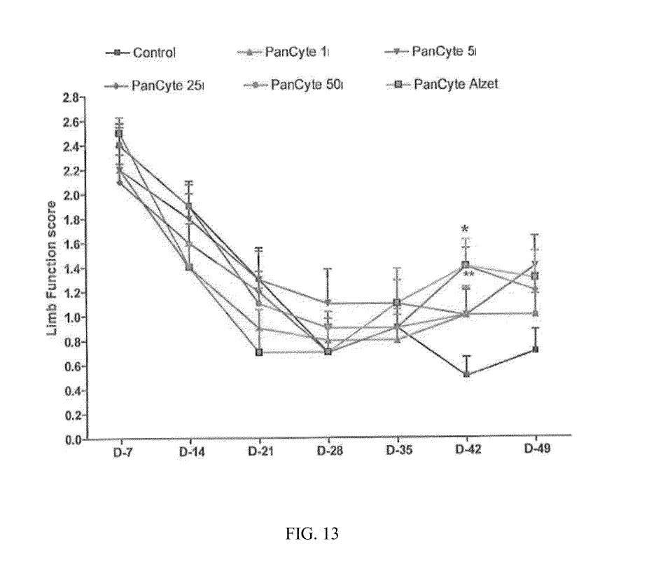

Semi-quantitative assessment of impaired use of the ischemic limb was performed on days 7 up to 49 by using graded functional scales. An improvement in limb function was found in the group treated with PanCyte 1 μg/kg (2F) and in continuous infusion group (6F) versus control group (1F) of animals on days 14 and 21 after hindlimb ischemia (see FIG. 13).

This example indicates that PanCyte can be an effective treatment for therapeutic angiogenesis. In order to assess the dose dependent therapeutic activity of PanCyte in ischemic tissue, an accepted mouse hind limb ischemia model was used. This example shows that subcutaneous administration of PanCyte restored blood flow in a dose dependent manner up to 84% of its normal values. Of the groups tested in this example, particularly good and early blood perfusion restoration was observed in animals treated with continuous PanCyte administration using Alzet pump.

### EQUIVALENTS AND SCOPE

Those skilled in the art will recognize, or be able to ascertain using no more than routine experimentation, many equivalents to the specific embodiments, described herein. The scope of the present invention is not intended to be limited to the above Description, but rather is as set forth in the appended claims.

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any cell type; any neuronal cell system; any reporter of synaptic vesicle cycling; any electrical stimulation system; any imaging system; any synaptic vesicle cycling assay; any synaptic vesicle cycle modulator; any method of use; etc.) can be excluded from any one or more claims, for any reason, whether or not related to the existence of prior art.

### INCORPORATION OF REFERENCES

All publications and patent documents cited in this application are incorporated by reference in their entirety to the same extent as though the contents of each individual publication or patent document were incorporated herein.

What is claimed is:

1. A method for treating peripheral vascular disease comprising a step of
administering a pharmaceutical composition comprising an angiotensin (1-7) peptide to an individual suffering from a peripheral vascular disease characterized by partial or complete blockage of blood flow to one or more tissues outside the heart and brain, wherein the angiotensin (1-7) peptide is administered in a therapeutically effective amount such that at least one symptom or feature of the peripheral vascular disease is reduced in intensity, severity, or frequency, or has delayed onset.

2. The method of claim 1, wherein the angiotensin (1-7) peptide comprises the naturally-occurring Angiotensin (1-7) amino acid sequence of Asp¹-Arg²-Val³-Tyr⁴-Ile⁵-His⁶-Pro⁷ (SEQ ID NO:1).

3. The method of claim 1, wherein the angiotensin (1-7) peptide is a functional equivalent of naturally-occurring Angiotensin (1-7).

4. The method of claim 3, wherein the functional equivalent is a linear peptide.

5. The method of claim 4, wherein the linear peptide contains 4-25 amino acids.

6. The method of claim 4, wherein the linear peptide is a fragment of the naturally-occurring Angiotensin (1-7).

7. The method of claim 4, wherein the linear peptide contains amino acid substitutions, deletions and/or insertions in the naturally-occurring Angiotensin (1-7).

8. The method of claim 7, wherein the linear peptide has an amino acid sequence of Asp¹-Arg²-Val³-Ser⁴-Ile⁵-His⁶-Cys⁷ (SEQ ID NO:5).

9. The method of claim 3, wherein the functional equivalent is a cyclic peptide.

10. The method of claim 9, wherein the cyclic peptide comprises a linkage between amino acids.

11. The method of claim 10, wherein the linkage is located at residues corresponding to positions Tyr⁴ and Pro⁷ in naturally-occurring Angiotensin (1-7).

12. The method of claim 10, wherein the linkage is a thioether bridge.

13. The method of claim 9, wherein the cyclic peptide comprises an amino acid sequence otherwise identical to the naturally-occurring Angiotensin (1-7) amino acid sequence of Asp¹-Arg²-Val³-Tyr⁴-Ile⁵-His⁶-Pro⁷ (SEQ ID NO:1).

14. The method of claim 9, wherein the cyclic peptide is a 4,7-cyclized angiotensin (1-7) with the following formula:

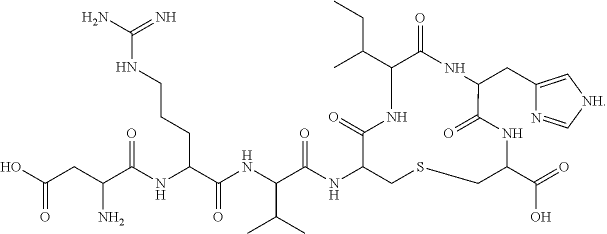 [SMILES: CCC(C)C1NC(=O)C(NC(=O)C(NC(=O)C(CCCNC(=N)N)NC(=O)C(N)CC(=O)O)C(C)C)CSCC(C(=O)O)NC(=O)C(Cc2c[nH]cn2)NC1=O]

15. The method of claim 1, wherein the angiotensin (1-7) peptide comprises one or more chemical modifications to increase protease resistance, serum stability and/or bioavailability.

16. The method of claim 1, wherein the one or more tissues outside the heart and brain comprise one or more limbs of the individual.

17. The method of claim 1, wherein the peripheral vascular disease is a peripheral artery disease.

18. The method of claim 16, wherein the peripheral artery disease is critical limb ischemia.

19. The method of claim 1, wherein the peripheral vascular disease is an acute ischemia, chronic ischemia, or diabetic vascular disease.

20. The method of claim 1, wherein the angiotensin (1-7) induces and/or increases angiogenesis and/or vascularization in the one or more tissues outside the heart and brain.

21. The method of claim 1, wherein the angiotensin (1-7) decreases and/or delays cell death in the one or more tissues outside the heart and brain.

22. The method of claim 1, wherein the angiotensin (1-7) increases and/or enhances cell survival in the one or more tissues outside the heart and brain.

23. The method of claim 1, wherein the therapeutically effective amount of the angiotensin (1-7) is sufficient to decrease partial or total blockage of blood flow to the one or more tissues outside the heart and brain.

24. The method of claim 1, wherein the therapeutically effective amount of the angiotensin (1-7) is sufficient to decrease or delay tissue damage in the one or more tissues outside the heart and brain.

25. The method of claim 1, wherein the angiotensin (1-7) is administered parenterally.

26. The method of claim 25, wherein the parenteral administration is selected from intravenous, intradermal, inhalation, transdermal (topical), subcutaneous, and/or transmucosal administration.

27. The method of claim 1, wherein the angiotensin (1-7) is administered orally.

28. The method of claim 1, wherein the angiotensin (1-7) is administered bimonthly, monthly, triweekly, biweekly, weekly, daily, or at variable intervals.

29. The method of claim 1, further comprising administering a pro-angiogenic agent in combination with the angiotensin (1-7).

30. A method for treating peripheral vascular disease comprising a step of
administering a pharmaceutical composition comprising an angiotensin-(1-7) receptor agonist to an individual suffering from a peripheral vascular disease characterized by partial or complete blockage of blood flow to one or more tissues outside the heart and brain,
wherein the angiotensin-(1-7) receptor agonist has a formula of 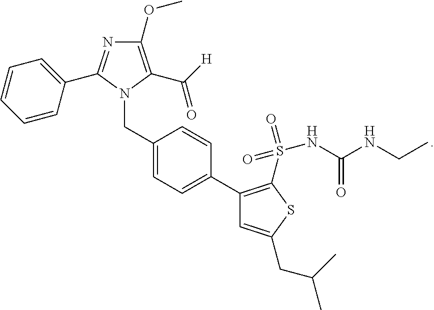 [SMILES: CCNC(=O)NS(=O)(=O)c1sc(CC(C)C)cc1-c1ccc(Cn2c(-c3ccccc3)nc(OC)c2C=O)cc1]
